SEQUENCE LISTING (46 sequences)

SEQ ID NO: 1 - DNA, 1788 bp, Homo sapiens
atgaccatgaccctccacaccaaagcatccgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 2 - protein, 595 aa, Homo sapiens [sequence omitted]

SEQ ID NO: 3 - DNA, 882 bp, Homo sapiens
aagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtc

SEQ ID NO: 4 - protein, 294 aa, Homo sapiens
KKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 5 - DNA, 1593 bp, Homo sapiens
atggatataaaaaactcaccatctagccttaattctccttcctcctacaactgcagtcaatccatcttacccctggagcacggctccatatacataccttcctcctatgtagacagccaccatgaatatccagccatgacattctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagtcctccatcacggggtccgagtgcagcccggcagaggacagtaaaagcaaagagggctcccagaacccacagtctcagtga

SEQ ID NO: 6 - protein, 530 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 7 - DNA, 1788 bp, Artificial Sequence
  Mutant human estrogen receptor alpha, T17-2
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacaggcagctggttcacatgatcaactgggcgaagagggtgccaggctttgtggatttgaccctccatgatcaggtccaccttctagaatgtgcctggctagagatcctgatgattgatctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 8 - protein, 595 aa, Artificial Sequence
  Synthetic Construct
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRQLVHMINWAKRVPGFVDLTLHDQVHLLECAWLEILMIDLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 9 - DNA, 1788 bp, Artificial Sequence
  Mutant human estrogen receptor alpha, Pg10
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacaggcagctggttcacatgatcaactgggcgaagagggtgccaggctttgtggatttgaccctccatgatcaggtccaccttctagaatgtgcctggctagagatcctgatgattgatctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagaatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 10 - protein, 595 aa, Artificial Sequence
  Synthetic Construct
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRQLVHMINWAKRVPGFVDLTLHDQVHLLECAWLEILMIDLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMENLYSMKCKNVVPLYDLLLEMLDAHRLHAPTSRGGASVEETDQSHLATAGSTSSHSLQKYYITGEAEGFPATV

SEQ ID NO: 11 - DNA, 1788 bp, Artificial Sequence
  Mutant human estrogen receptor alpha, Pg10-1
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacaggcagctggttcacatgatcaactgggcgaagagggtgccaggctttgtggatttgaccctccatgatcaggtccaccttctagaatgtgcctggctagagatcctgatgattgatctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagaatctgtacagcatgaagtgcaagaacgtggtgccccactatgacctgctgctggagatgctggacgcccaccgcctacatgcgcccactagccgtggaggggcatccgtggaggagacggaccaaagccacttggccactgcgggctctacttcatcgcattccttgcaaaagtattacatctcgggggaggcagagggtttccctgccacagtctga

SEQ ID NO: 12 - protein, 595 aa, Artificial Sequence
  Synthetic Construct
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRQLVHMINWAKRVPGFVDLTLHDQVHLLECAWLEILMIDLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMENLYSMKCKNVVPHYDLLLEMLDAHRLHAPTSRGGASVEETDQSHLATAGSTSSHSLQKYYISGEAEGFPATV

SEQ ID NO: 13 - DNA, 1788 bp, Artificial Sequence
  Mutant human estrogen receptor alpha, Pg10-16
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacaggcagctggttcacatgatcaactgggcgaagagggtgccaggctttgtggatttgaccctccatgatcaggtccaccttctagaatgtgcctggctagagatcctgatgattgatctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagaatctgtacagcttgaagtgcaagaacgtggtgcccccctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 14 - protein, 595 aa, Artificial Sequence
  Synthetic Construct
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRQLVHMINWAKRVPGFVDLTLHDQVHLLECAWLEILMIDLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMENLYSLKCKNVVPPYDLLLEMLDAHRLHAPTSRGGASVEETDQSHLATAGSTSSHSLQKYYITGEAEGFPATV

SEQ ID NO: 15 - DNA, 1788 bp, Artificial Sequence
  Mutant human estrogen receptor alpha, T17
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacaggcagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctga

SEQ ID NO: 16 - protein, 595 aa, Artificial Sequence
  Synthetic Construct
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRQLVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 17 - protein, 583 aa, Acanthopagrus schlegelii
MYPEDSRVSGGVATVDFLEGTYDYAAPTPAPTPLYSHSTPGYYSAPLDAHGPPSDGSLQSLGSGPNSPLVFVPSSPRLSPFMHPPTHHYLETTSTPIYRSSVPSSQHSASREDQCGTSDDSYSVGESGAGAGAAGFEMAKEMRFCAVCSDYASGYHYGVWSCEGCKAFFKRSIQGHNDYMCPATNQCTIDRNRRKSCQACRLRKCYEVGMMKGGVRKDRGRVLRRDKRRTGTSDRDKASKGLEHRTAPPQDRRKHISSSAAGGGGKSSVISMPPDQVLLLLQGAEPPMLCSRQKVNRPYTEVTVMTLLTSMADKELVHMIAWAKKLPGFLQLSLHDQVQLLESSWLEVLMIGLIWRSIHCPGKFIFAQDFILDRSEGDCVEGMAEIFDMLLATASRFRMLKLKPEEFVCLKAIVLLNSGAFSFCTGTMEPLHDGAAVQNMLDTITDALIHHINQSGCTAQQQSRRQAQLLLLLSHIRHMSNKGMEHLYSMKCKNKVPLYDLLLEMLDAHRVHRPDRPHETWSQADREPPFTSRNNRGSGGGGGSSSAGSTSGTRVSLENPTGPGVLQYGRSAPSAPHPMKPTE

SEQ ID NO: 18 - protein, 587 aa, Alligator mississippiensis
MTMTLHTKTSGVTLLHQIQGTELETLSRPQLKIPLDRSLSEMYVESNKTGIFNYPEGTTYDFATAAPVYSSTSLSYAPTSESYGSSSLGGFHSLNNVPPSPVVFLQTAPQLSPFIHHHSQQVPYYLENDQSGFGMREAAPSTFYRPGADSRRQSGRERMSSTSEKTSLSMESTKETRYCAVCNDYASGYHYGVWSCEGCKAFFKRSIQGHNDYMCPATNQCTIDKNRRKSCQACRLRKCYEVGMMKGGIRKDRRGGRMLKQKRQREEQDARNGETATAEMRTPTLWTSPLVIKHTKKNSPALSLTAEQMVSALLEAEPPIVYSEYDPNRPFNEASMMTLLTNLADRELVHMINWAKRVPGFVDLTLHDQVHLLECAWLEILMIGLVWRSVEHPGKLLFAPNLLLDRNQGKCVEGMVEIFDMLLATAARFRMMNLQGEEFVCLKSIILLNSGVYTFLSSTLKSLEEKDYIHRVLDKITDTLIHLMAKSGLSLQQQHRRLAQLLLILSHIRHMSNKGMEHLYNMKCKNVVPLYDLLLEMLDAHRLHAPAARNAAQVEEETRLTTASASSHSLQSFYINNREDENLQNTI

SEQ ID NO: 19 - protein, 582 aa, Astatotilapia burtoni
MYPEESRGSGGVATVDFLEGSYDYAAPTPAPTPLYSHSTTGCYSAPLDAHGPPSDGSLQSLGSGTTSPLVFVPSSPRLSPFMHPPSHHYLETTSTPVYRSSHQPVPRDDQCGTRDEAYGLGELGAGAGGFEMTKETRFCAVCSDYASGYHYGVWSCEGCKAFFKRSIQGHNDYMCPATNQCTIDKNRRKSCQACRLRKCYEVGMMKGGMRKDRGRVLRREKRRAYDRDKPAKDLPHTKAPPHDGRKHATSSSSTSGGGGRSSLNSIPPDQVLLLLQGAEPPTLCSRQKMNQPYTEVTMMTLLTSMADKELVHMIAWAKKLPGFLQLSLHDQVLLLESSWLEVLMIGLIWRSIHCPGKLIFAQDLILDRTEGTCVEGMAEIFDMLLATASRFRMLKLKPEEFVCLKAIILLNSGAFSFCTGTMEPLHDSAAVQHMLDTITDALIFHISQLGCSAQHQSRRQAQLLLLLSHIRHMSNKGMEHLYSMKCKNKVPLYDLLLEMLDAQRIHRPVKPSQSWSQGDRDSPNTSSSGGGGSDDEGTSSAGSSSGPQGNHESPRCENLSRAPTGPGVLQYRGSHSDCTPIL

SEQ ID NO: 20 - protein, 596 aa, Bos taurus
MTMTLHTKASGMALLHQIQANELEPLNRPQLKIPLERPLGEVYMDSSKPAVYNYPEGAAYDFNAAAPASAPVYGQSGLPYGPGSEAAAFGANGLGAFPPLNSVSPSPLVLLHPPPQPLSPFLHPHGQQVPYYLENESSGYAVREAGPPAYYRPNSDNRRQGGRERLASTSDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRNEAVPSGDMRAANLWPSPIMIKHTKKNSPVLSLTADQMISALLEAEPPIIYSEYDPTRPFSEASMMGLLTNLADRELVHMINWAKRVPGFVDLALHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLRSLEEKDHIHRVLDKITDTLIHLMAKAGLTLQQQHRRLAQLLLILSHFRHMSNKGMEHLYSMKCKNVVPLYDLLLEMLDAHRLHAPANFGSAPPEDVNQSQLAPTGCTSSHSLQTYYITGEAENFPSTV

SEQ ID NO: 21 - protein, 587 aa, Caiman crocodilus
MTMTLHTKTSGVTLLHQIQGTELETLSRPQLKIPLDRSLSEMYVENNKTGIFNYPEGTTYDFATAAPVYSSTSLSYAPTSESYGSSSLGGFHSLNNVPPSPVVFLQTAPQLSPFVHHHSQQVPYYLENDQSGFGMREAASSTFYRPSADSRHQSGRERMSSTSEKASLSMESTKETRYCAVCNDYASGYHYGVWSCEGCKAFFKRSIQGHNDYMCPATNQCTIDKNRRKSCQACRLRKCYEVGMMKGGIRKDRRGGRMLKQKRQREEQDARNGETATAEMRTPTLWTSPLVIKHTKKNSPALSLTAEQMVSALLEAEPPIVYSEYDPNRPFNEASMMTLLTNLADRELVHMINWAKRVPGFVDLTLHDQVHLLECAWLEILMIGLVWRSMEHPGKLLFAPNLLLDRNQGKCVEGMVEIFDMLLATAARFRMMNLQGEEFVCLKSIILLNSGVYTFLSSTLKSLEEKDYIHRVLDKITDTLIHLMAKSGLSLQQQHRRLAQLLLILSHIRHMSNKGMEHLYNMKCKNVVPLYDLLLEMLDAHRLHAPAARNAAQVEEETRLTTASASSHSLQSFYINNREDENLQNTI

SEQ ID NO: 22 - protein, 353 aa, Cavia porcellus
RKCYDVGMIKGGIRKDRRGGRMLKYKRQRDDEERRNEMGPSGDMRGSNLWPSPLVIKHTKKNSPALSLTADQMVSALMDAEPPLLYSEYDAVKPFSEASMMGLLTNLADRELVHMINWAKRVPGFGDLTLHDQVHLLECAWLEILMIGLIWRSMEHPGKLLFAPNLILDRNQGKCVEGMVEIFDMLLATSTRFRMMNLQGEEFVCLKSIILLNSGMYTFLSSTLKSLEEKDHIHRVLDKIIDTLIHLMAKAGLTLQQQHRRLAQLLLILSHIRHMSNKGVEHLYNMKCKNVVPLYNLLLEMLEAHRLNTSSNPMGGSPEEPSQSQLATIGSSSAHSLQTYYISQEAESFPNTI

SEQ ID NO: 23 - protein, 581 aa, Chrysophrys major
MYPEDSRGSGGVATVDFLEGTYDYAAPTPAPTPLYSHSTPGYYSAPLDAHGPPSDGSLQSLGSGPNSPLVFVPSSPRLSPFMHPPTHHYLETTSTPVYRSSVPSSQQSVSREDQCGTSDDSYSVGESGAGALAAGFEIAKEMRFCAVCSDYASGYHYGVWSCEGCKAFFKRSIQGHNDYMCPATNQCTIDRNRRKSCQACRLRKCYEVGMMKGGMRKDRGRVLRRDKQRTGTSDRDKASKGLEHRTAPPQDRRKHISSSAGGGGGKSSMISMPPDQVLLLLQGAEPPMLCSRQKLNRPYTEVTMMTLLTSMADKELVHMIAWAKKLPGFLQLSLHDQVQLLESSWLEVLMIGLIWRSIHCPGKLIFAQDLILDRSEGDCVEGMAEIFDMLLATASRFRMLKLKPEEFVCLKAIILLNSGAFSFCTGTMEPLHDGAAVQNMLDTITDALIHHINQSGCSAQQQSRRQAQLLLLLSHIRHMSNKGMEHLYSMKCKNKVPLYDLLLEMLDAHRIHRADRPAETWSQADREPPFTSRNSSGGGGGGGGGSSSAGSTSGPRVSHESPTSPGVLQYGGSRSECTHIL

SEQ ID NO: 24 - protein, 589 aa, Coturnix japonica
MTMTLHTKASGVTLLHQIQGTELETLSRPQLKIPLERSLSDMYVESNKTGVFNYPEGATYDFGTTAPVYGSTTLSYAPTSESFGSSSLAGFHSLNNVPPSPVVFLQTAPQLSPFIHHHSQQVPYYLENEQGSFGMRETAPPAFYRPSSDNRRHSIRERMSSASEKGSLSMESTKETRYCAVCNDYASGYHYGVWSCEGCKAFFKRSIQGHNDYMCPATNQCTIDKNRRKSCQACRLRKCYEVGMMKGGIRKDRRGGRMMKQKRQREEQESRNGEASSTELRAPTLWTSPLVVKHNKKNSPALSLTAEQMVSALLEAEPPIVYSEYDPNRPFNEASMMTLLTNLADRELVHMINWAKRVPGFVDLTLHDQVHLLECAWLEILMIGLVWRSMEHPGKLLFAPNLLLDRNQGKCVEGMVEIFDMLLATAARFRMMNLQGEEFVCLKSIILLNSGVYTFLSSTLKSLEERDYIHRVLDKITDTLIHFMAKSGLSLQQQHRRLAQLLLILSHIRHMSNKGMEHLYNMKCKNVVPLYDLLLEMLDAHRLHAPAARSAAPMEEENRSQLTTAPASSHSLQSFYINSKEEESMQNTI

SEQ ID NO: 25 - protein, 569 aa, Danio rerio
MYPKEEHSAGGISSSVNYLDGAYEYPNPTQTFGTSSPAEPASVGYYPAPPDPHEEHLQTLGGGSSSPLMFAPSSPQLSPYLSHHGGHHTTPHQVSYYLDSSSSTVYRSSVVSSQQAAVGLCEELCSATDRQELYTGSRAAGGFDSGKETRFCAVCSDYASGYHYGVWSCEGCKAFFKRSIQGHNDYVCPATNQCTIDRNRRKSCQACRLRKCYEVGMMKGGIRKDRGGRSVRRERRRSSNEDRDKSSSDQCSRAGVRTTGPQDKRKKRSGGVVSTLCMSPDQVLLLLLGAEPPAVCSRQKHSRPYTEITMMSLLTNMADKELVHMIAWAKKVPGFQDLSLHDQVQLLESSWLEVLMIGLIWRSIHSPGKLIFAQDLILDRSEGECVEGMAEIFDMLLATVARFRSLKLKLEEFVCLKAIILINSGAFSFCSSPVEPLMDNFMVQCMLDNITDALIYCISKSGASLQLQSRRQAQLLLLLSHIRHMSNKGMEHLYRMKCKNRVPLYDLLLEMLDAQRFQSSGKVQRVWSQSEKNPPSTPTTSSSSSNNSPRGGAAAIQSNGACHSHSPDP

SEQ ID NO: 26 - protein, 594 aa, Equus caballus
MTMTLHTKASGMALLHQIQGNELETLNLPQFKIPLERPLGEVYVESSKPPVYDYPEGAAYDFNAAAAASASVYGQSGLAYGPGSEAAAFGANGLGGFPPLNSVSPSQLMLLHPPPQLSPYLHPPGQQVPYYLENEPSGYSVCEAGPQAFYRPNADNRRQGGRERLASSGDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRNEAGPSGDRRPANFWPSPLLIKHTKKISPVLSLTAEQMISALLDAEPPVLYSEYDATRPFNEASMMGLLTNLADRELVHMINWAKRVPGFVDLSLHDQVHLLECAWLEILMIGLVWRSMEHPGKLLFAPNLLLDRNQGKCVEGMVEIFDMLLATSSRLRMMNLQGEEFVCLKSIILLNSGVYTFLSSTLKSLEEKDHIHRVLDKMTDTLIHLMAKAGLTLQQHRRLAQLLLILSHIRHMSNKGMEHLYSMKCKNVVPLYDLLLEMLDAHRLHAPANHGGAPMEETNQSQLATTGSTSPHSMQTYYITGEAEGFPNTI

SEQ ID NO: 27 - protein, 573 aa, Fundulus heteroclitus
MYPEESRGSGGVAAVDFLEGTYDYATPTPAPTPLYSHSTTGYYSAPLDAQGPPSDGSLHSLGSGPTSPLVFVPTSPRLSLFMHAPSQHYLETASTPVYRSSHQPASREDQCDTRDEACSVGELGAGAGAGAAAGGFEMAKETRFCAVCSDYASGYHYGVWSCEGCKAFFKRSIQGHNDYMCPATNQCTIDRNRRKSCQACRLRKCYEVGMMKGGVRKERGRVLRRDKRRTAISDREKAVKGLEPKTSPHQDKRRRGSALGGDRSSVASLPSEQVLLLFQGAEPPILCSRQKLSRPYTEVTMMTLLTSMADKELVHMIAWAKKLPGFLQLALHDQVLLLESSWLEVLMIGLIWRSIHCPGKLIFAQDLILDRNEGDCVEGMTEIFDMLLATASRFRMLKLKPEEFVCLKAIILLNSGAFSFCTGTMEPLHDSVAVQNMLDTITDALIHHISQSGFSVQQQARRQAQLLLLLSHIRHMSNKGMEHLYSMKCKNKVPLYDLLLEMLDAHRHHPVKPSQDGKSPPSTSSFGAGCEGGSSSAGSSSGPRGSGDNLMRIHSAPGVLQYGGSRSDCAQVL

SEQ ID NO: 28 - protein, 574 aa, Halichoeres tenuispinis
MYPEESRGSGGVGTVDFLEGTYDYTAPTPAPTLYSLSTQGYYSAALDTHGQPSDSSIQSLGSGPSSPLVFVPSSPRLSPFMHLPSHHYLETSSTPVYRSSVSSSQQSISREEHCGTSDESYSMGESGAGAAAGCFEMAKEMRYCAVCSDYASGYHYGVWSCEGCKAFFKRSIQGHNDYMCPATNQCTIDRNRRKSCQACRLRKCYEVGMMKGGVRKDRGRVLRRDKRRTGTSDKDNGSKDREQRTVPPQGRRKHGSSVGGGKSPVISMPPDQVLLLLQGAEPPILCSRQKLSRPYTEVTMMTLLTSMTDRELVHMIAWAKKLPGFLQLTLHDQVQLLESSWLEVLMIGLIWRSIHCPGKLIFAQDLILDRSEGDCVEGMAEIFDMLLATTSRFRMLKLKPEEFVCLKAIILLNSGAFSFCTGTMEPLHDNEAVQNMLDIITDALIHHISQSGCSAHQQSRRQAQLLLLLSHIRHMSNKGMEHLYSMKCKNKVPLYDLLLEMLDAHRLHRPDRPAESWYQTDREPAYSSSATTTNDNSSSSPAGSRASQESPNRPPTGHSVLQFGGSRSDCTHIL

SEQ ID NO: 29 - protein, 458 aa, Halichoeres trimaculatus
SDESYGMGESGAGAAAGCFEMAKEMRYCAVCSDYASGYHYGVWSCEGCKAFFKRSIQGHNDYMCPATNQCTIDRNRRKSYQACRLRKCYEVGMMKGGARKDRGRVLRRDKRRTCTSDKDKGSKERDERTAPPQAGGNTATVWEENPQWISMPPDQVLLLLQGAETPILYSRQKLSRPYTEVTMMTLLTSMADRELVHMIAWAKKLPGFLQLTHHDQVQLLESSWLEVLMIGLIWRSIHCRGKHIFAQDLILDRNEGDCVEGMAEIFDMLLATTSPFRMLKLKPEEFVCLKAIVLLNSGAFSFCTGTMEPLHDSAPVQDMLDIITDALIHHISQSGCSAHQQSRRQAQLLLLLSHIRHMSNKGMEHLYSMKCKNKVPLYDLLLEMLDAHRLHRPDRPAESWSQTDGEPAYSSSATTTNDSNNNSSSAGSRAGHEGPNKPPTSPGVLQYGGSRSDCTHVL

SEQ ID NO: 30 - protein, 581 aa, Ictalurus punctatus
MYPEEEQRTTGGISSTAHYLDGTFNYTTNPDATNSSVDYYSVAPEPQEENLQPLPNGSSSPPVFVPSSPQLSPFLGHPPAGQHTAQQVPYYLEPSGTSIYRSSVLASAGSRVELCSAPGRQDVYTAVGASGPSGASGPSGAIGLVKEIRYCSVCSDYASGYHYGVWSCEGCKAFFKRSIQGHNDYVCPATNQCTIDRNRRKSCQACRLRKCYEVGMMKGGFRKERGGRIIKHNRRPSGLKERERGYSKAQSGSDVREALPQDGQSSSGIGGGVADVVCMSPEQVLLLLLRAEPPTLCSRQKHSRPYSELTIMSLLTNMADRELVHMIAWAKKVPGFQDLSLHDQVQLLESSWLEILMIGLIWRSIYTPGKLIFAQDLILDKSEGECVEGMAEIFDMLLATVARFRTLKLKSEEFVCLKAIILLNSGAFSFCSSPVEPLRDGFMVQCMMDNITDALIYYISQSGISVQLQSRRQAQLLLLLSHIRHMSYKGMEHLYSMKCKNKVPLYDLLLEMLDAHRLRPLGKVPRIWADRVSSSPTTTATTPTTNTTTTTTTTTHHPSNGSTCPADLPSNPPGPGQSPSP

SEQ ID NO: 31 - protein, 627 aa, Micropterus salmoides
MCKRQSPAQSKQPCGTVLRPRIGPAFTELETLSPQHPSPPLRAPLSDMYPEESRGSGGGATVDFLEGTYDYVAPTPVPTPLYSHSGYYSAPLDAQGPPSDGSLQSLGSGPTSPLVFVPSSPRLSPFMHPPSHHYLETTSTPVYRSSVLSSQQPVPREDQCATSDESYCVGESGAGAGGFEMAKEMRFCAVCSDYASGYHYGVWSCEGCKAFFKRSIQGHNDYMCPATNQCTIDRNRRKSCQACRLRKCYEVGMMKGGVRKDRGRVLRRDKRRAGTNDRDKASKDLEYRTVPPQDRRKHSSSSAGGGGGKSSVTGMSPDQVLLLLQGAEPPMLCSRQKLSRPYTEVTIMTLLTSMADKELVHMITWAKKLPGFLQLSLHDQVQLLESSWLEVLMIGLIWRSIHCPGKLIFAQDLILDRNEGDCVEGFVEIFDMLLATASRFRMLKLKPEEFVCLKAIILLNSGAFSFCTGTMEPLHNSVEVHNMLDTITDALIHHISQSGCSAQQQSRRQAQLLLLLSHIRHMSNKGMEHLYSMKCKNKVPLYDLLLEMLDAHRIHRPDRPAQFWSQADGEPPFITVNNCNSSSNGGVSSSVGSSSGPRVSHESPSRGPTGPGVLQYGGSRSDCTHIL

SEQ ID NO: 32 - protein, 599 aa, Mus musculus
MTMTLHTKASGMALLHQIQGNELEPLNRPQLKMPMERALGEVYVDNSKPTVFNYPEGAAYEFNAAAAAAAAASAPVYGQSGIAYGPGSEAAAFSANSLGAFPQLNSVSPSPLMLLHPPPQLSPFLHPHGQQVPYYLENEPSAYAVRDTGPPAFYRSNSDNRRQNGRERLSSSNEKGNMIMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDLEGRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKGMEHLYNMKCKNVVPLYDLLLEMLDAHRLHAPASRMGVPPEEPSQTQLATTSSTSAHSLQTYYIPPEAEGFPNTI

SEQ ID NO: 33 - protein, 643 aa, Ovis aries
SLPSHCLSPLLQAHGTFLERRSSSRVAGRLLSPLPRGETVCAGPRLTMTMTLHTKASGMALLHQIQANELEPLNRPQLKIPLERPLGEMYVDSSKPAVYNYPEGAAYDFNAAAAASAPVYGQSGLPYGPGSEAAAFGANGLGAFPPLNSVSPSPLVLLHPPPQPLSPFLHPHGQQVPYYLENEPSGYAVREAGPPAYYRPNSDNRRQGGRERLASTSDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRNEAVPSGDMRATNLWPSPIMIKHTKKNSPVLSLTADQMISALLEAEPPIIYSEYDPTRPFSEASMMGLLTGLADRELVHMINWAKRVPGFVDLALHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLRSLEEKDHIHRVLDKITDTLIHLMAKAGLTLQQQHRRLAQFLLLLSHFRHMSNKGMEHLYSMKCKNVVPLYDLLLEMLDAHRLHAPANFGSTPPEDVNQSQLATTGCTSSHSLQTYYITGEAENFPSTV

SEQ ID NO: 34 - protein, 620 aa, Oncorhynchus masou
MLVRQSHTQISKPLGAPLRSRTTLESHVISPTKLSPQQPTTPNSNMYPEETRGGGGAAAFNYLDGGYDYTAPAQGPAPLYYSTTPQDAHGPPSDGSMQSLGSSPTGPLVFVSSSPQLSPQLSPFLHPPSHHGLPSQSYYLETSSTPLYRSSVVTNQLSASEEKLCIASDRQQSYSAAGSGVRVFEMANETRYCAVCSDFASGYHYGVWSCEGCKAFFKRSIQGHNDYMCPATNQCTMDRNRRKSCQACRLRKCYEVGMVKGGLRKDRGGRVLRKDKRYCGPAGDREKPYGDLEHRTAPPQDGVRNSSSSLNGGGGWRGPRITMPPEQVLFLLQGAEPPALCSRQKVARPYTEVTMMTLLTSMADKELVHMIAWAKKVPGFQELSLHDQVQLLESSWLEVLMIGLIWRSIHCPGKLIFAQDLILDRSEGDCVEGMAEIFDMLLATVSRFRMLKLKPEEFLCLKAIILLNSGAFSFCSNSVESLHNSSAVESMLDNITDALIHHISHSGASVQQQPRRQAQLLLLLSHIRHMSNKGMEHLYSIKCKNKVPLYDLLLEMLDGHRLQSPGKVAQAGEQTEGPSTTTTTSTGSSIGPMRGSQDTHIRSPGVLQYGSPSSDQMPIP

SEQ ID NO: 35 - protein, 578 aa, Paralichthys olivaceus
MYPEESRGSGGAATVDFLEGTYDYAAPTPAQTPLYSHSTSGYYSAPLDAHGPPSDGSRHSLGSGPTSPHVYVPSSPRLSPFMHPPSHHYLETTATSVYRSSQQPVTREDHCGPRDESFSVGETGAAAGAEGFEMAKETRFCAVCSDYASGYHYGVWSCEGCKAFFKRSIQGHNDYMCPATNQCTIDRNRRKSCQACRLRKCYEVGMMKGGVRKDRSHVLRRDKRRAGTNDRDKASKDQDHKTVPLQDGRKSSSSTAGGKSSVTAMLPDQVLVLLQGAEPPILCSRQKLNQPYTEVTMMTLLTSMADRELVHMIAWAKKLPGFLQLSLHDQVQLLESSWLEVLMIGLIWRSIHCPGKLIFAQDLILDRNEGNCVEGMAEIFDMLLATASRFRMLKLKSEEFFCLKAIILLNSGSFSFCTGTMEPLHNTAAVQDMLETITDALIHHISQSGCPVQQQWRRQAQLLLLLSHIRHMSNKGMEHLYSMKCKNKVPLYDLLLEMLDAHCLHRPARPAQSWLQADREPSAAGNNNNNSSSIIISGGGSSSASSGHRGSQESPSRATTGPSVLQHGGSRPDCTHIL

SEQ ID NO: 36 - protein, 579 aa, Sparus aurata
MYPEDSRVSGGVATVDFLEGTYDYAAPTPAPTPLYSHSTPGYYSAPLDAHGPPSDGSLQSLGSGPNSPLVFVPSSPHLSPFMQPANHHYLETTSTPIYSVPSSQHSVSREDQCGTSDDSYSVGESGAGAGAAGFEMAKEMRFCAVCSDYASGYHYGVWSCEGCKAFFKRSIQGHNDYMCPATNQCTIDRNRRKSCQACRLRKCYEVGMMKGGVRKDRGRVLRRDKRRTGTSDRDKASKGLEHRTAPPQDRRKHISSSAGGGGGKSSVISMPPDQVLLLLRGAEPPMLCSRQKVNRPYTEVTVMTLLTSMADKELVHMIAWAKKLPGFLQLSLHDQVQLLESSWLEVLMIGLIWRSIHCPGKLIFAQDLILDRSEGDCVEGMAEIFDMLLATASRFRMLKLKPEEFVCLKAIILLNSGAFSFCTGTMEPLHDSAAVQNMLDTITDALIHHINQSGCSAQQQSRRQAQLLLLLSHIRHMSNKGMEHLYSMKCKNKVPLYDLLLEMLDAHRVHRPDRPAETWSQADREPLFTSRNSSSSSGGGGGGSSSAGSTSGPQVNLESPTGPGVLQLRVHPHPMKPTE

SEQ ID NO: 37 - protein, 587 aa, Taeniopygia guttata
MTLHTKTSGVTLLHQIQGTELETLSRPQLKIPLERSLSDMYVETNKTGVFNYPEGATYDFGTTAPVYSSTTLSYAPTSESFGSSSLAGFHSLNSVPPSPVVFLQTAPHWSPFIHHHSQQVPYYLENDQGSFGMREAAPPAFYRPNSDNRRHSIRERMSSANEKGSLSMESTKETRYCAVCNDYASGYHYGVWSCEGCKAFFKRSIQGHNDYMCPATNQCTIDKNRRKSCQACRLRKCYEVGMMKGGIRKDRRGGRVMKQKRQREEQDSRNGEASSTELRAPTLWASPLVVKHNKKNSPALSLTAEQMVSALLEAEPPLVYSEYDPNRPFNEASMMTLLTNLADRELVHMINWAKRVPGFVDLTLHDQVHLLECAWLEILMIGLVWRSMEHPGKLLFAPNLLLDRNQGKCVEGMVEIFDMLLATAARFRMMNLQGEEFVCLKSIILLNSGVYTFLSSTLKSLEEKDYIHRVLDKITDTLIHLMAKSGLSLQQQHRRLAQLLLILSHIRHMSNKGMEHLYNMKCKNVVPLYDLLLEMLDAHRLHAPAARSAAPMEEENRSQLTTASASSHSLQSFYINSKEEENMQNTL

SEQ ID NO: 38 - protein, 585 aa, Tilapia nilotica
MYPEESRGSGGVATVDFLEGTYDYAAPTPAPTPLYSHSTTGCYSAPLDAHGPLSDGSLQSLGSGPTSPLVFVPSSPRLSPFMHPPSHHYLETTSTPVYRSSHQPVPREDQCGTRDEAYSVGELGAGAGGFEMTKDTRFCAVCSDYASGYHYGVWSCEGCKAFFKRSIQGHNDYMCPATNQCTIDKNRRKSCQACRLRKCYEVGMMKGGMRKDRGRVLRREKRRACDRDKPAKDLPHTRASPQDGRKRAMSSSSTSGGGGRSSLNNMPPDQVLLLLQGAEPPILSSRQKMSRPYTEVTIMTLLTSMADKELVHMITWAKKLPGFLQLSLHDQVLLLESSWLEVLMIGLIWRSIQCPGKLIFAQDLILDRNEGTCVEGMAEIFDMLLATASRFRVLKLKPEEFVCLKAIILLNSGAFSFCTGTMEPLHDSAAVQHMLDTITDALIFHISHLGCSAQQQSRRQAQLLLLLSHIRHMSNKGMEHLYSMKCKNKVPLYDLLLEMLDAHRIHRPVKPFQSWSQGDRDSPTASSTSSSGGGGGDDEGASSAGSSSGPQGSHESPRRENLSRAPTGPGVLQYRGSHSDCTRIP

SEQ ID NO: 39 - protein, 427 aa, Xenopus laevis
MSSANDKGPPSMESTKETRFCAVCSDYASGYHYGVWSCEGCKAFFKRSIQGHNDYMCPATNQCTIDKNRRKSCQACRLRKCYEVGMMKGGIRKDRRGGRMLKHKQQKEEPEQKNDVNPSEIRTASIWVNPSVKSMKLSPVLSLTAEQLISALMEAEPPIVYSEHDSTKPLSEASMMTLLTNLADKELVHMINWAKRVPGFVDLTLHDQVHLLECAWLEILMVGLIWRSVEHPEKLSFAPNLLLDRNQGRCVEGLVEIFDMLVTTATRFRMMRLHGEEFICLKSIILLNSGVYTFLSSTLESLEDTDLIHIILDKIIDTLVHFMAKSGLSLQQQQRRLAQLLLILSHIRHMSNKGMEHLYSMKCKNVVPLYDLLLEMLDAHRIHTPKDKTTTQEEESRSPLSTTVNGASPCLQPFYKNTEEVSLQSTV

SEQ ID NO: 40 - protein, 9 aa, Artificial Sequence
  Sequence motif of homing endonucleases
LAGLIDADG

SEQ ID NO: 41 - DNA, 18 bp, Artificial Sequence
  I-SceI recognition sequence
tagggataacagggtaat

SEQ ID NO: 42 - DNA, 18 bp, Artificial Sequence
  GLC1A gene target sequence
cagggggagctgggcacc

SEQ ID NO: 43 - DNA, 18 bp, Artificial Sequence
  Synthetic target sequence
tagggataacagggcaat

SEQ ID NO: 44 - DNA, 18 bp, Artificial Sequence
  Synthetic target sequence
tagggggaacagggcaat

SEQ ID NO: 45 - DNA, 18 bp, Artificial Sequence
  Synthetic target sequence
tagggggagctgggcaat

SEQ ID NO: 46 - DNA, 18 bp, Artificial Sequence
  Synthetic target sequence
tagggggagctgggcacc

(19) United States
(12) Patent Application Publication
(10) Pub. No.: US 2006/0183159 A1
(43) Pub. Date: Aug. 17, 2006

(54) Method for engineering a protein by in vitro coevolution

(76) Inventors: Huimin Zhao, Champaign, IL (US); Zhilei Chen, Champaign, IL (US)

Correspondence Address: Jane Massey Licata;Licata & Tyrrell P.C., 66 E. Main Street, Marlton, NJ, 08053, US

(21) Appl. No.: 11/355,544

(22) Filed: Feb. 16, 2006

Related U.S. Application Data
(60) Provisional application No. 60/654,269, filed on Feb. 17, 2005.

(51) Int. Cl. C40B 40/10; C07K 14/72; G01N 33/53; C07H 21/04; C12P 21/06

(52) U.S. Cl. 435/7.1; 530/350; 435/69.1; 435/320.1; 435/325; 536/23.5

(57) ABSTRACT

The present invention relates to compositions and methods for generating proteins with novel functions. The methods employ an in vitro coevolution approach that, in a stepwise manner, generates one or more intermediate functions. A pathway containing one or more analog molecules corresponding to the intermediate functions, and a target molecule, corresponding to the target function are designed and used to select mutant proteins exhibiting the target function.

### INTRODUCTION

This application claims benefit of U.S. Provisional Patent Application Ser. No. 60/654,269, filed Feb. 17, 2005, the content of which is incorporated herein by reference in its entirety.

This invention was made with government support under Grant Number BES-0348107, awarded by The National Science Foundation. The Government may have certain rights to this invention.

### BACKGROUND OF THE INVENTION

Two different, yet complementary approaches have been developed to design, modify and engineer naturally occurring proteins at the molecular level. These approaches include rational design and directed evolution (Brannigan & Wilkinson (2002) Nat. Rev. Mol. Cell Biol. 3:964-970; Penning & Jez (2001) Chem. Rev. 101:3027-3046; Arnold (2001) Nature 409:253-257). Rational design involves the rational alterations of selected residues in a protein via site-directed mutagenesis, and requires detailed knowledge of protein folding, structure, function, and dynamics. In contrast, directed evolution mimics the process of natural evolution in the test tube, involving repeated cycles of creating molecular diversity by random mutagenesis and/or gene recombination and screening/selecting the functionally improved variants. Both approaches have been successfully used to engineer a wide variety of protein functions, such as stability, activity, affinity, selectivity and pH profiles (Brannigan & Wilkinson (2002) supra; Penning & Jez (2001) supra; Arnold (2001) supra; Arnold (1998) Acct. Chem. Res. 31:125-131; Schmidt-Dannert (2001) Biochemistry 40:13125-13136). Although these methods have been used to engineer existing protein functions, there is a need in the art for methods that can be used to create novel protein functions (Brannigan & Wilkinson (2002) supra; Penning & Jez (2001) supra; Arnold (2001) supra; Lo Surdo, et al. (2004) Nat. Struct. Mol. Biol. 11:382-383; Bolon, et al. (2002) Curr. Opin. Chem. Biol. 6:125-129).

### SUMMARY OF THE INVENTION

The present invention relates to methods for creating proteins with novel functions. The methods utilize an in vitro coevolution approach that mimics the process of natural coevolution in the test tube. The methods involve design of a pathway containing one or more analog molecules for use in combination with directed evolution to generate a protein capable of carrying out a novel function. Typically, the pathway includes at least one analog molecule that differs from a base molecule by at least a single structural transformation, and a second analog molecule that differs from the first analog molecule by at least a single structural transformation. The second analog molecule can be another intermediate in the pathway or a target molecule. Directed evolution is applied in a stepwise fashion to generate at least a first library and a second library of mutant proteins capable of interacting with the first analog molecule and/or the target analog molecule. The use of the methods described herein permits the generation of proteins with novel function that would be difficult to obtain using rational design or directed evolution approaches.

In other embodiments, nucleic acids, proteins and fragments thereof, with novel functions are provided. For example, in some embodiments, novel receptor proteins are provided. Sources of receptor proteins suitable for use in the methods and compositions described herein include, but are not limited to, nuclear hormone receptors. In other embodiments, novel enzymes are provided. Sources of enzymes suitable for use in the methods and compositions described herein include, but are not limited to, kinases, phosphatases, oxidoreductases, transferases, hydrolases, lyases, isomerases, ligases, and homing endonucleases.

Additionally, expression vectors and cells expressing the various compositions described herein are provided.

### BRIEF DESCRIPTION OF THE DRAWINGS

FIGS. 1A-1D depict dose-response profiles of the wild-type estrogen receptor hERαLBD (WT) and mutant estrogen receptor proteins (T17, T17-2, Pg10, Pg10-1, Pg10-16) to different ligands. FIG. 1A depicts the response to E₂. FIG. 1B depicts the response to testosterone. FIG. 1C depicts the response to progesterone. FIG. 1D depicts the response to corticosterone.

FIG. 2 depicts an exemplary in vitro co-evolution approach for generating a pathway composed of enzymes capable of denitrogenation of carbazole.

### DETAILED DESCRIPTION OF THE INVENTION

It has now been found that by mimicking natural coevolution in vitro, proteins with novel functions can be generated. Provided herein are methods and compositions for engineering novel proteins. The methods use an in vitro coevolution approach in which the novel function is divided into one or more intermediate functions amenable to classical directed evolution. A pathway containing one or more analog molecules corresponding to the intermediate functions in combination with a target molecule, corresponding to a target function, are designed and used to select mutants exhibiting the desired function. Single and/or double mutants expressing an intermediate function are selected and used in subsequent rounds of directed evolution until one or more mutants exhibiting the target function is identified.

In vitro coevolution differs from rational design and directed evolution methodologies because mutants with multiple simultaneous or synergistic mutations are generated. In contrast to proteins generated using rational design methodologies in which the mutations are typically limited to a particular region, the multiple simultaneous or synergistic mutations generated using in vitro coevolution are located throughout the protein. Moreover, unlike methodologies based on directed evolution, in vitro coevolution does not require the screening of a large number of possible mutants, i.e., >10¹³ to identify mutants exhibiting the desired function. Thus, in vitro coevolution is used to create variants with novel functions that require the acquisition of multiple simultaneous or synergistic mutations in order to be expressed.

As used herein, “novel function” means that the binding interactions or activity of a target protein is altered in some detectable, observable and/or measurable way as compared to the binding interactions or activity of a wild-type or normal protein. In particular embodiments, the novel function is readily detectable, observable and/or measurable as a phenotype of a cell expressing the protein with the novel function. For example, in some embodiments, small molecule-protein pairs are generated in which the protein cannot be activated by endogenous small molecules. As another example, small molecule-enzyme pairs are generated in which the enzyme recognizes a molecule that is not an endogenous substrate. As a further example, orthogonal ligand-receptor pairs are generated in which the receptor cannot be activated by endogenous small molecules and the ligand cannot activate endogenous receptors.

In some embodiments, proteins with “altered functions” are generated. By “altered function” herein is meant any characteristic or attribute of a protein that can be selected or detected and compared to the corresponding property of a wild-type or variant protein, e.g., designed protein or proteins created by mutagenic methods such as combinatorial cassette, oligonucleotide-directed mutagenesis, error-prone PCR, DNA shuffling, and random priming synthesis. These properties include, but are not limited to cytotoxic activity, oxidative stability, substrate specificity, substrate binding or catalytic activity, thermal stability, alkaline stability, pH activity profile, resistance to proteolytic degradation, kinetic association (Kₒₙ) and dissociation (K_{off}) rate, protein folding, inducing an immune response, ability to bind to a ligand, ability to bind to a receptor, ability to be secreted, ability to be displayed on the surface of a cell, ability to oligomerize, ability to signal, ability to stimulate cell proliferation, ability to inhibit cell proliferation, ability to induce apoptosis, ability to be modified by phosphorylation or glycosylation, ability to treat disease.

In other embodiments, phenotypic changes can be induced in cells expressing the mutant protein(s). Examples of possible phenotypic changes include, gross physical changes such as changes in cell morphology, cell growth, cell viability, adhesion to substrates or other cells, and cellular density. Other examples include changes in the expression of one or more RNAs, proteins, lipids, hormones, cytokines, or other molecules. As another specific example, the changes can include changes in the equilibrium state (i.e., half-life) of one or more RNAs, proteins, lipids, hormones, cytokines, or other molecules. As another example, the changes can include changes in the localization of cellular constituents, such as RNAs, proteins, lipids, hormones, cytokines, or other molecules. As a further example, the changes can include changes in the activity of cellular constituents, such as changes in the bioactivity or specific activity of one or more RNAs, proteins, lipids, hormones, cytokines, receptors, or other molecules. Other changes that can be detected and/or measured include changes in phosphorylation; secretion of ions and other small molecules such as cytokines, hormones, growth factors, or other molecules; alterations in cellular membrane potential, polarization, integrity or transport; changes in infectivity, susceptibility, latency, adhesion, and uptake of viruses and bacterial pathogens; changes in carbon or nitrogen source utilization.

In accordance with the instant methods, the protein and target molecule are coevolved. Typically, each molecule in the pathway differs from the molecule that precedes it and follows it by a single structural change. The structural change can be achieved using a single analog molecule. Alternatively, two or more analog molecules can be used to effect the single structural changes. For example, in some embodiments, between two to ten analog molecules are used. In other embodiments, ten or more analog molecules are used.

In some embodiments, the analog molecules are structurally related to an endogenous base molecule present in a cell, e.g., an enzyme substrate, a ligand or an antigen. In other embodiments, the analog molecules are related to base molecules that are not endogenous to a cell, such as haptens, transition state analogs, or drugs. Typically, the analog molecules are molecules that are not capable of activating or activating only slightly (i.e., less than 10% of wild-type activation) the protein used to generate the first library or mutant proteins.

In some embodiments, two or more structural changes occur between the base molecule and the target molecule. In these embodiments, an analog molecule corresponding to each structural change is used. As exemplified herein, testosterone and progesterone were used as analogs of base molecule E₂ to derive target molecule corticosterone.

In other embodiments, more than one analog molecule is used to effect each structural change. In some embodiments, three, four, five, six, seven, eight, nine, ten or more structural changes occur between the base molecule and the target molecule. In these embodiments, an analog molecule corresponding to each structural change is used. In other embodiments, more than one analog molecule is used to effect each structural change. Thus, any number of analog molecules can be used to effect one or more structural changes between the base molecule and the target molecule, provided that a stepwise pathway between the base molecule and the target molecule is created.

As used herein, “structural change” refers to a change in a substituent group, a change in the oxidation state of the molecule, and/or a change in the number of carbon or heteroatoms present. The structural change can result in the addition of a substituent group, replacement of one substituent group by another, a change in the number of saturated and unsaturated bonds, the replacement of a substituent group by a hydrogen atom, and/or the addition or removal of a carbon atom or a heteroatom.

“Substituent” refers to any atom or group replacing a hydrogen of a base molecule. The nature of these substituent groups can vary broadly. Non-limiting examples of suitable substituent groups include branched, straight-chain or cyclic alkyls, mono- or polycyclic aryls, branched, straight-chain or cyclic heteroalkyls, mono- or polycyclic heteroaryls, halos, branched, straight-chain or cyclic haloalkyls, hydroxyls, oxos, thioxos, branched, straight-chain or cyclic alkoxys, branched, straight-chain or cyclic haloalkoxys, trifluoromethoxys, mono- or polycyclic aryloxys, mono- or polycyclic heteroaryloxys, ethers, alcohols, sulfides, thioethers, sulfanyls (thiols), imines, azos, azides, amines (primary, secondary and tertiary), nitriles (any isomer), cyanates (any isomer), thiocyanates (any isomer), nitrosos, nitros, diazos, sulfoxides, sulfonyls, sulfonic acids, sulfamides, sulfonamides, sulfamic esters, aldehydes, ketones, carboxylic acids, esters, amides, amidines, formadines, amino acids, acetylenes, carbamates, lactones, lactams, glucosides, gluconurides, sulfones, ketals, acetals, thioketals, oximes, oxamic acids, oxamic esters, etc., and combinations of these groups. Substituent groups bearing reactive functionalities can be protected or unprotected, as is well-known in the art.

In the context of the present invention, “alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc., and the like. Where specific levels of saturation are intended, the nomenclature “alkanyl,” “alkenyl” and/or “alkynyl” is used. “Lower alkyl” refers to alkyl groups having from 1 to 6 carbon atoms. In some embodiments, alkyl groups contain from 6 to 30 carbon atoms, or from 6 to 25 carbon atoms, or from 6 to 20 carbon atoms, or from 6 to 15 carbon atoms, or from 8 to 30 carbon atoms, or from 8 to 25 carbon atoms, or from 8 to 20 carbon atoms, or from 8 to 15 carbon atoms, or from 12 to 30 carbon atoms, or from 12 to 25 carbon atoms, or from 12 to 20 carbon atoms.

“Alkanyl” by itself or as part of another substituent refers to a saturated branched, straight-chain or cyclic alkyl derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl; and the like.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group can be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl, cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like.

“Alkyldiyl” by itself or as part of another substituent refers to a saturated or unsaturated, branched, straight-chain or cyclic divalent hydrocarbon group having the stated number of carbon atoms (i.e., C1-C6 means from one to six carbon atoms) derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to, methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl; butyldiyls such as butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl; and the like. Where specific levels of saturation are intended, the nomenclature alkanyldiyl, alkenyldiyl and/or alkynyldiyl is used. Where it is specifically intended that the two valencies are on the same carbon atom, the nomenclature “alkylidene” is used. A “lower alkyldiyl” is an alkyldiyl group having from 1 to 6 carbon atoms. In some embodiments, the alkyldiyl groups are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenes).

“Alkylene” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The location of a double bond or triple bond, if present, in a particular alkylene is indicated in square brackets. Typical alkylene groups include, but are not limited to, methylene (methano); ethylenes such as ethano, etheno, ethyno; propylenes such as propano, prop[1]eno, propa[1,2]dieno, prop[1]yno; butylenes such as butano, but[1]eno, but[2]eno, buta[1,3]dieno, but[1]yno, but[2]yno, buta[1,3]diyno; and the like. Where specific levels of saturation are intended, the nomenclature alkano, alkeno and/or alkyno is used. In some embodiments, the alkylene group is (C1-C6) or (C1-C3) alkylene. In other embodiments, the alkylene group contains straight-chain saturated alkano groups, e.g., methano, ethano, propano, butano, and the like.

“Cycloalkyl” by itself or as part of another substituent refers to a cyclic version of an “alkyl” group. Typical cycloalkyl groups include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —O—O—, —S—S—, —O—S—, —NRR, —═N—N═—, —N═N—, —N═N—NRR, —PR—, —P(O)₂—, —POR⁰—, —O—P(O)₂—, —SO—, —SO₂—, —SnR⁵¹R⁵²—, and the like, where R can independently be hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group is from 5-20 membered heteroaryl, more preferably from 5-10 membered heteroaryl. Preferred heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In some embodiments, the heteroarylalkyl group is a 6-30-membered heteroarylalkyl, e.g., the alkanyl, alkenyl or alkynyl moiety of the heteroarylalkyl is 1-10-membered and the heteroaryl moiety is a 5-20-membered heteroaryl. In other embodiments, the heteroarylalkyl group is a 6-20-membered heteroarylalkyl, e.g., the alkanyl, alkenyl or alkynyl moiety of the heteroarylalkyl is 1-8-membered and the heteroaryl moiety is a 5-12-membered heteroaryl.

“Parent heteroaromatic ring system” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In some embodiments, the aryl group is (C5-C15) aryl. In other embodiments the aryl group is (C5-C10). In other embodiments, the aryl group can comprise phenyl and/or naphthyl.

“Halogen” or “Halo” by themselves or as part of another substituent, unless otherwise stated, refer to fluoro, chloro, bromo and iodo.

“Haloalkyl” by itself or as part of another substituent refers to an alkyl group in which one or more of the hydrogen atoms is replaced with a halogen. Thus, the term “haloalkyl” is meant to include monohaloalkyls, dihaloalkyls, trihaloalkyls, up to perhaloalkyls. For example, the expression “(C1-C2) haloalkyl” includes fluoromethyl, difluoromethyl, trifluoromethyl, 1-fluoroethyl, 1,1-difluoroethyl, 1,2-difluoroethyl, 1,1,1-trifluoroethyl, perfluoroethyl, etc.

The above-defined groups can include prefixes and/or suffixes that are commonly used in the art to create additional well-recognized substituent groups. As examples, “alkyloxy” or “alkoxy” refers to a group of the formula —OR, “alkylamine” refers to a group of the formula —NHR and “dialkylamine” refers to a group of the formula —NRR, where each R is independently an alkyl. As another example, “haloalkoxy” or “haloalkyloxy” refers to a group of the formula —OR′, where R′ is a haloalkyl.

In accordance with the instant invention one, two, or more of the structural changes occurring in a pathway can involve a change in a substituent group. For example, in some embodiments, one substituent group is used to replace another, i.e., an alcohol replaces a carboxylic acid. As another example, a substituent group is added or removed on one or more of the analog and/or target molecules in the pathway, i.e., one or more alkyl, alkanyl, alkenyl, alkynl, alkyldiyl, alkylene, cyclalkyl, heteroalkyl, heteroaryl groups, etc., as defined herein, is added or removed. In other embodiments, a hydrogen atom replaces a substituent group.

One, two, or more of the structural changes occurring in a pathway can also involve a change in the oxidation state of one or more of the molecules of a pathway. “Oxidation state” refers to a change in the type and/or number of bonds in the base molecule. The oxidation state can vary between two or more of the molecules of the pathway. The molecules of the pathway can be a mixture of single, double and triple bonds. The bonds can be formed between carbon atoms, between heteroatoms and between carbon and heteroatoms. For example, the base molecule is composed of carbon-carbon single bonds, and one or more of the analog molecules and the target molecule is composed of at least one carbon-carbon double bond. As another example, the base molecule is a mixture of carbon-carbon single bonds and carbon-carbon double bonds, and one or more of the analog molecules and/or the target molecule is composed of at least one carbon-carbon triple bond. As a further example, the base molecule is composed of at least one carbon-carbon double bond, and at least one or more of the analog molecules and/or target molecules is composed of carbon-carbon single bonds. In other embodiments, single, double and triple bonds are removed. Moreover, substituents composed of single, double and triple bonds, including but not limited to, alkyl, alkanyl, alkenyl, alkynl, alkyldiyl, alkylene group are added or removed at different steps along the pathway.

One, two, or more of the structural changes occurring in a pathway can further involve a change in the number of carbon or heteroatoms of a base molecule. For example, the base molecule is composed of six carbon atoms and the target molecule is composed of fifteen carbon atoms. As another example, the base molecule is composed of an aryl hydrocarbon group and the target molecule is composed of a heteroaryl group. As a further example, the base molecule is composed of a fifteen carbon group and the target molecule is composed of a six carbon group. Similarly, the base molecule is composed of a heteroaryl group and the target molecule is composed of an aryl group.

Thus, any combination of substituent groups, oxidation states, addition and deletion of carbon and/or heteroatoms can be used, provided that a stepwise pathway between the base molecule and the target molecule is created.

Virtually any existing protein can be used as the starting point for the generation of a novel target molecule/protein pair. The protein can be a wild-type protein or a mutant protein that exhibits an altered function as compared to the wild-type protein. For example, the mutant protein can exhibit enhanced catalytic activity as compared to the wild-type protein. As another specific example, the mutant protein can exhibit altered substrate specificity as compared to the wild-type protein. Thus, the protein used to generate the first library of mutant proteins can be any protein, which when mutated as described herein, can be used to generate at least a first mutant protein exhibiting a novel function.

Following identification of at least one mutant protein from the first library of mutant proteins, at least a second library of mutant proteins is generated using directed evolution. In some embodiments, a plurality of secondary libraries is generated. For example, if two mutant proteins are identified from the first library, each mutant protein, independently of the other can be used to generate a secondary library of mutant proteins. The libraries are screened for mutant proteins that are activated by the second analog. For example, in some embodiments, the second mutant protein is activated by the second analog, but not by the first analog. In other embodiments, the second mutant protein is activated by the first and the second analog, but not by the base molecule. In yet other embodiments, the second mutant protein is activated by the first analog, the second analog, and the base molecule.

Proteins of the invention can be provided from any source. The sample containing the protein can be provided from nature or it can be synthesized or supplied from a manufacturing process. For example, the proteins can be obtained from an organism, including prokaryotes and eukaryotes, with proteins from bacteria, fungi, viruses, extremophiles such as the archaebacteria, insects, fish, mammals, humans, and birds all possible. The protein does not need to be naturally occurring. For example, the protein can be a designed protein, or a protein selected by a variety of methods including, but not limited, to directed evolution (Farinas, et al. (2001) Curr. Opin. Biotechnol. 12:545-551; Morawski, et al. (2001) Biotechnol. Bioengineer. 76:99-107; Stemmer (1994) Nature 370(6488):389-91; Ness, et al. (2000) Adv. Protein. Chem. 55:261-92), DNA shuffling (e.g., technologies available from MAXYGEN®, ENCHIRA, DIVERSA®) or ribosome display (Hanes, et al. (2000) Meth. Enzymol. 328:404-430; Hanes and Pluckthun (1997) Proc. Natl. Acad. Sci. USA 94:4937-4942; Roberts and Szostak (1997) Proc. Natl. Acad. Sci. USA 94:12297-302).

Proteins suitable for use in the methods and compositions described herein include, but are not limited to, industrial and pharmaceutical proteins which interact with base or analog molecules as disclosed herein. As used in the context of the present invention, a protein is said to “interact” with a base, analog, or target molecule in the sense that the molecule binds, activates, inhibits, or is a substrate or ligand for the protein. In some embodiments, known proteins with known or predictable structures, including mutant proteins, are used. Examples of known proteins with known or predictable structures include, but are not limited to cytokines, hormones and extracellular signaling moieties; transcription factors and other DNA binding proteins; antibodies; antigens and trojan horse antigens; cell surface receptors; cytoskeletal proteins; enzymes; protein domains and motifs; etc.

Cytokines of the invention include, e.g., IL-1Ra (+receptor complex), IL-1 (receptor alone), IL-1a, IL-1b (including variants and or receptor complex), IL-2, IL-3, IL-4, IL-5, IL-6, IL-8, IL-10, IFN-β, INF-γ, IFN-α-2a, IFN-α-2B, TNF-α, CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration Inhibitory Factor, Human Glycosylation-Inhibiting Factor, Human RANTES, Human Macrophage Inflammatory Protein 1 Beta, human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor A, Vascular Endothelial growth factor (VEGF), acidic Fibroblast growth factor, basic Fibroblast growth factor, Endothelial growth factor, Nerve growth factor, Brain-Derived Neurotrophic Factor, Ciliary Neurotrophic Factor, Platelet Derived Growth Factor, Human Hepatocyte Growth Factor, Fibroblast Growth Factor (including but not limited to alternative splice variants, abundant variants, and the like), Glial Cell-Derived Neurotrophic Factor, and hemopoietic receptor cytokines (including but not limited to erythropoietin, thrombopoietin, and prolactin), APM1, and the like.

Extracellular signaling moieties which can be coevolved include, but are not limited to, sonic hedgehog, protein hormones such as chorionic gonadotrophin and leutenizing hormone.

Transcription factors and other DNA binding proteins of the invention, include but are not limited to, histones, p53, myc, PIT1, NFkB AP1, JUN, KD domain, homeodomain, heat shock transcription factors, stat, zinc finger proteins (e.g., zif268).

Antibodies, antigens, and trojan horse antigens of use as starting proteins, include, but are not limited to, immunoglobulin super family proteins, e.g., CD4 and CD8, Fc receptors, T-cell receptors, MHC-I, MHC-II, CD3, and the like. Immunoglobulin-like proteins are also embraced by the present invention. Such proteins include, e.g., fibronectin, pkd domain, integrin domains, cadherins, invasins, cell surface receptors with Ig-like domains, intrabodies, anti-Her/2 neu antibody (e.g., HERCEPTIN®), anti-VEGF, anti-CD20 (e.g., RITUXAN®), etc.

Receptors embraced by the present invention include, but are not limited to, the extracellular region of human tissue factor cytokine-binding region of Gp130; G-CSF receptor; erythropoietin receptor; fibroblast growth factor receptor; TNF receptor; IL-1 receptor; IL-1 receptor/IL1Ra complex; IL-4 receptor; INF-γ receptor alpha chain; MHC Class I; MHC Class II; T cell receptor; insulin receptor; tyrosine kinase receptors; human growth hormone receptor; G-protein coupled receptors; ABC Transporters/Multidrug resistance proteins such as MRP or MDR1; hormone receptors such as human estrogen receptor α (SEQ ID NOs:1 and 2; GENBANK Accession No. NM_{—}000125), human estrogen receptor β (SEQ ID NOs:5 and 6; GENBANK Accession No. NM_{—}001437) human progesterone receptor (GENBANK Accession No. NM_{—}000926), human androgen receptor (GENBANK Accession No. NM_{—}000044 or NM_{—}001011645), human glucocorticoid receptor (GENBANK Accession No. NM_{—}000176), human mineralocorticoid receptor (GENBANK Accession No. M16801), human thyroid hormone receptor α (GENBANK Accession No. NM_{—}199334), human thyroid hormone receptor β (GENBANK Accession No. NM_{—}000461); human retinoid receptors such as human retinoid X receptor β (GENBANK Accession No. NM_{—}021976), human retinoid X receptor α (GENBANK Accession No. NM_{—}002957), human retinoic acid receptor a (GENBANK Accession No. NM_{—}000964), human retinoic acid receptor β (GENBANK Accession No. NM_{—}000965 or NM_{—}016152); human vitamin D receptor (GENBANK Accession No. J03258); human peroxisome proliferator-activated receptor a (GENBANK Accession No. Y07619); human peroxisome proliferator-activated receptor γ (GENBANK Accession No. L40904); human peroxisome proliferator-activated receptor (GENBANK Accession No. L02932); liver X receptor; farnesoid X receptor; and ecdysone receptor; aquaporins; transporters; RAGE (receptor for advanced glycan end points); TRK-A; TRK-B; TRK-C; hemopoietic receptors; and the like.

Enzymes as starting proteins for coevolution include, but are not limited to, hydrolases such as proteases/proteinases, synthases/synthetases/ligases, decarboxylases/lyases, peroxidases, ATPases, carbohydrases, lipases; isomerases such as racemases, epimerases, tautomerases, or mutases; transferases, kinases, reductases/oxidoreductases, hydrogenases, polymerases, phosphatases, and proteasomes anti-proteasomes, (e.g., MLN341), thioredoxins, homing endonucleases, and the like.

Protein domains and motifs are intended to include, but are not limited to, SH-2 domains, SH-3 domains, Pleckstrin homology domains, WW domains, SAM domains, kinase domains, death domains, RING finger domains, Kringle domains, heparin-binding domains, cysteine-rich domains, leucine zipper domains, zinc finger domains, nucleotide binding motifs, transmembrane helices, and helix-turn-helix motifs. Additionally, ATP/GTP-binding site motif A, Ankyrin repeats, fibronectin domain, Frizzled (fz) domain, GTPase binding domain, C-type lectin domain, PDZ domain, Homeobox domain, Krueppel-associated box (KRAB), cellulose binding domain, leucine zipper, DEAD and DEAH box families, ATP-dependent helicases, HMG1/2 signature, DNA mismatch repair proteins mutL/hexB/PMS1 signature, thioredoxin family active site, annexins repeated domain signature, clathrin light chains signatures, mycotoxin signatures, Staphylococcal enterotoxins/Streptococcal pyrogenic exotoxins signatures, Serpins signature, cysteine proteases inhibitors signature, chaperones, heat shock domains, WD domains, EGF-like domains, immunoglobulin domains, immunoglobulin-like proteins, and the like.

Once a pathway has been created, directed coevolution is used to generate the libraries of mutant proteins used in the methods described herein. By “directed coevolution” is meant the generation and selection or screening of a pool of mutated nucleic acid molecules having sufficient diversity for a nucleic acid molecule encoding a protein with a novel or altered function to be present and interact with one or more analog and/or target molecules of a pathway. Any number of libraries can be generated using the methods described herein provided that one or more mutants with the desired novel function can be identified. For example, in some embodiments, a first library and a second library are generated. In other embodiments, a first, second, third and fourth library are generated. In still other embodiments, four or more libraries are generated. As another specific example, in some embodiments, the number of libraries corresponds to the number of analog and target molecules of the pathway. For example, if the pathway has two analog molecules and one target molecule, three libraries are generated. In another embodiment, the number of libraries generated is greater than the number of analog and target molecules of the pathway. For example, if the pathway has two analog molecules and one target molecule, four or more libraries can be generated.

The template nucleic acid for the first library is generally a nucleic acid molecule or fragment thereof encoding a wild-type or mutant protein. The template can be used in any of the amplification techniques described herein to generate a first library of mutant proteins. The first library of mutant proteins is screened, using any one of the screens described herein, to identify one or more mutant proteins capable of interacting with the first analog molecule in the pathway. Mutant proteins capable of interacting with the first analog molecule in the pathway are isolated, and each of the nucleic acid molecules encoding the proteins are used as templates to generate one or more secondary (i.e., second) libraries of mutant proteins. Depending on the level of interaction between the first analog molecule and the mutant protein used to generate the secondary library, the secondary library can be screened to identify one or more mutant proteins capable of interacting with the first analog molecule or with the next molecule in the pathway. Depending on the design of the pathway, the next molecule in the pathway can be an analog molecule or a target molecule.

The level of interaction between the mutant protein(s) and the various molecules of the pathway can be selected by the user, depending, in part, on the particular application. For example, in some embodiments, if the target molecule is a drug, a mutant protein that responds only to the drug and not to the other molecules of the pathway, e.g., the base and analog molecules, may be desired. In other embodiments, mutant proteins that respond to the different molecules of the pathway may be desired. For example, if the pathway has a base molecule, a first analog molecule, a second analog molecule and a target molecule, it may be desirable to isolate a mutant protein that responds to the first analog molecule and not to the base molecule, the second analog molecule or the target molecule. As another example, it may be desirable to isolate a mutant protein that responds to the second analog molecule, but not to the base molecule, the first analog molecule or the target molecule. As another example, it may be desirable to isolate a mutant protein that responds to the first and second analog molecule as well as the target molecule. Thus, mutant proteins exhibiting different levels of activation to one or more of the molecules in a pathway can be generated using the methods disclosed herein.

In some embodiments, the level of activation by the base, analog, and target molecules is expressed as an EC₅₀ values in nM. Generally, EC₅₀ values range from 10 to greater than 10,000 nM. For example, the EC₅₀ for a wild-type protein can be 500 nM for a base molecule and greater than 10,000 nM for an analog or target molecule. Accordingly, in some embodiments, the EC₅₀ for a mutant protein generated using the methods described herein is greater than 10,000 nM for the base molecule and in the range of 20 to 5000 nM for an analog or target molecule.

In other embodiments, the level of activation by the base, analog, and target molecules is expressed as an efficacy measurement. Efficacy, given as a percent, is defined as the maximum increase in activation relative to the increase in activation of wild-type with a given concentration of a base molecule. For example, in some embodiments, the efficacy for a wild-type protein is 100% for the base molecule and from 10 to 25% for an analog or target molecule. In contrast, the efficacy for a mutant protein can be from 0 to 25% for the base molecule and from 10 to 100% for an analog or target molecule.

The libraries of mutant proteins can be generated using any one of the PCR amplification techniques described herein. In addition, other amplification techniques can also be used to generate the libraries of mutant proteins. For example, in some embodiments, error-prone PCR is used. “Error-prone PCR” refers to a process for performing PCR under conditions where the copying fidelity of the DNA polymerase is lowered, such that a high rate of point mutations is obtained along the entire length of the PCR product. See, e.g., See U.S. Pat. Nos. 5,605,793; 5,811,238; and 5,830,721.

In some embodiments “assembly PCR” is used. “Assembly PCR” refers to a process that involves the assembly of a PCR product from a mixture of small DNA fragments. A large number of different PCR reactions occur in parallel in the same vial, with the products of one reaction priming the product off another. See e.g., U.S. Pat. No. 6,806,048.

In some embodiments, “DNA shuffling” is used. “DNA shuffling” refers to forced homologous recombination between DNA molecules of different but highly related DNA sequences in vitro, caused by random fragmentation of the DNA molecule based on sequence homology, followed by fixation of the crossover by primer extension. See, e.g., WO 00/42561 and WO 01/70947.

In some embodiments, sequences derived from introns are used to mediate specific cleavage and ligation of discontinuous nucleic acid molecules to create libraries of novel genes and gene products as described in U.S. Pat. Nos. 5,498,531 and 5,780,272.

In some embodiments, libraries composed of ribonucleic acids encoding a novel gene product or novel gene products are created by mixing splicing constructs composed of an exon and 3′ and 5′ intron fragments. See e.g., U.S. Pat. No. 5,498,531.

In other embodiments, DNA libraries are created by mixing DNA/RNA hybrid molecules that contain intron-derived sequences that are used to mediate specific cleavage and ligation of the DNA/RNA hybrid molecules such that the DNA molecules are covalently linked to form novel DNA molecules as described in WO 00/40715 and WO 00/17342, and U.S. Pat. No. 6,150,141.

In some embodiments, multiple amplification reactions with pooled oligonucleotides, composed of mutant protein sequences created by the assembly of gene fragments generated from a nucleic acid template are used. See, e.g., U.S. Pat. No. 6,403,312.

Examples of other suitable mutagenesis techniques, include, but are not limited to, exon shuffling (U.S. Pat. No. 6,365,377; Kolkman & Stemmer (2001) Nature Biotechnology 19:423-428), family shuffling (Crameri, et al. (1998) Nature 391:288-291; U.S. Pat. No. 6,376,246), RACHITT™ (Coco, et al. (2001) Nature Biotechnology 19:354-359; WO 02/06469), STEP and random priming of in vitro recombination (Zhao, et al. (1998) Nature Biotechnology 16:258-261; Shao, et al. (1998) Nucl. Acids Res. 26:681-683); exonucleases-mediated gene assembly (U.S. Pat. Nos. 6,352,842 and 6,361,974), GENE SITE SATURATION MUTAGENESIS™ (U.S. Pat. No. 6,358,709), GENE REASSEMBLY™ (U.S. Pat. No. 6,358,709) and SCRATCHY (Lutz, et al. (2001) Proc. Natl. Acad. Sci. USA 98:11248-11253), DNA fragmentation methods (Kikuchi, et al. (1999) Gene 236:159-167), and single-stranded DNA shuffling (Kikuchi, et al. (2000) Gene 243:133-137).

Although these methods are intended to introduce random mutations throughout the gene, those skilled in the art will appreciate that specific regions of the gene can be mutated, and others left untouched, either by isolating and combining the mutated region with the unmodified region, e.g., by cassette mutagenesis (see, WO 01/75767; Kim & Mass (2000) Biotechniques 28:196-198; Lanio & Jeltsch (1998) Biotechniques 25:958-965; Ge & Rudolph (1997) Biotechniques 22:28-30; Ho, et al. (1989) Gene 77:51059). Alternatively, in vitro or in vivo recombination can be employed (see, e.g., WO 02/10183; Abécassis, et al. (2000) Nucl. Acids Res. 28:e88).

A number of other methods an also be used to generate the libraries disclosed herein. For example, in some embodiments, oligonucleotide-directed mutagenesis can be used. Oligonucleotide-directed mutagenesis refers to a process that allows for the generation of site-specific mutations in any cloned DNA segment of interest. See, e.g., Ehrlich (1989) PCR Technology, Stockton Press; Oliphant, et al. (1986) Gene 44:177-183; Hermes, et al. (1988) Science 241:53-57; Knowles (1990) Proc. Natl. Acad. Sci. USA 87:696-700. As another specific example, classical site-directed mutagenesis, e.g. QUICKCHANGE™ commercially available from STRATAGENE® can be used to generate the libraries described herein. As another example, cassette mutagenesis can be used. In some embodiments, cassette mutagenesis includes the creation of DNA molecules from restriction digestion fragments using nucleic acid ligation, and the random ligation of restriction fragments (Kikuchi, et al., (1999) supra). Additionally, cassette mutagenesis can be performed using randomly-cleaved nucleic acids (Kikuchi et al. (2000) supra), by PCR-ligation PCR mutagenesis (see, for example, Ali & Steinkasserer (1995) Biotechniques 18:746-750), by seamless gene engineering using RNA- and DNA-overhang cloning (Coljee, et al. (2000) Nature Biotechnology 18:789-791), by ligation-mediated gene construction, by homologous or non-homologous random recombination (U.S. Pat. Nos. 6,368,861; 6,423,542; 6,376,246; 6,368,861; 6,319,714; and WO 00/42561; WO 00/42561; WO 00/42560; WO 00/42560; WO 00/42559; WO 00/18906; WO 00/18906; and WO 00/18906), or in vivo using recombination between flanking sequences (WO 02/10183; Abécassis, et al. (2000) Nucl. Acids Res. 28:e88 for examples). In addition, regions of the template oligonucleotide encoding the wild-type protein can be mutated in E. coli lacking correct mismatch repair mechanisms, (e.g., E. coli strain XLmutS commercially available from STRATAGENE®), or by using phage display techniques to evolve a library (e.g., Long-McGie, et al. (2000) Biotechnol. Bioeng. 68:121-125).

In addition to the PCR methods outlined herein, other amplification and gene synthesis methods can be used to generate the libraries of mutant proteins. For example, the library genes can be “stitched” together using pools of oligonucleotides with polymerases (and optionally or solely) ligases. These resulting variable sequences can then be amplified using any number of amplification techniques, including, but not limited to, polymerase chain reaction (PCR), strand displacement amplification (SDA), nucleic acid sequence-based amplification (NASBA), ligation chain reaction (LCR) and transcription-mediated amplification (TMA). In addition, there are a number of variations of PCR which can also find use in the invention, including quantitative competitive PCR (QC-PCR), arbitrarily-primed PCR (AP-PCR), immuno-PCR, Alu-PCR, PCR single-strand conformational polymorphism (PCR-SSCP), reverse transcriptase PCR (RT-PCR), biotin-capture PCR, vectorette PCR, panhandle PCR, and PCR-select cDNA subtraction, among others. Furthermore, by incorporating the T7 polymerase initiator into one or more oligonucleotides, IVT amplification can be performed.

Library of proteins are produced by culturing a host cell transformed with nucleic acid molecules, preferably an expression vector containing nucleic acid molecules encoding a library of proteins, under the appropriate conditions to induce or cause expression of the library of proteins. The conditions appropriate for library protein expression will vary with the choice of the expression vector and the host cell, and can be ascertained by one skilled in the art through routine experimentation. For example, the use of constitutive promoters in the expression vector requires optimizing the growth and proliferation of the host cell, while the use of an inducible promoter requires the appropriate growth conditions for induction. In addition, in some embodiments, the timing of the harvest is important. For example, the baculovirus systems used in insect cell expression are lytic viruses, and thus harvest time selection can be crucial for product yield.

A wide variety of appropriate host cells can be used to produce and screen the mutant libraries, including yeast, bacteria, archaebacteria, fungi, insect, plant and animal cells, including mammalian cells. Of particular interest are Drosophila melanogaster cells, Saccharomyces cerevisiae and other yeasts, E. coli, Bacillus subtilis, Streptococcus cremoris, Streptococcus lividans, SF9 cells, C129 cells, 293 cells, Neurospora, BHK cells, CHO cells, COS cells, HeLa cells, fibroblasts, Schwanoma cell lines, immortalized mammalian myeloid and lymphoid cell lines, Jurkat cells, mast cells and other endocrine and exocrine cells, and neuronal cells. Suitable host cells can be readily obtained from the ATCC cell line catalog. In some embodiments, the cells are genetically engineered to contain exogenous nucleic acid molecules, for example, to contain target molecules.

In some embodiments, the library of proteins is expressed in vitro using cell-free translation systems. Several commercial sources are available for this including, but not limited, to Roche RAPID TRANSLATION SYSTEM™, PROMEGA® TNT® system, the NOVAGEN® ECOPRO™ system, the AMBION® PROTEINSCRIPT-PRO™0 system. In vitro translation systems derived from both prokaryotic (e.g., E. coli) and eukaryotic (e.g., Wheat germ, Rabbit reticulocytes) cells are available and can be selected based on the expression levels and functional properties of the protein of interest. Both linear (as derived from a PCR amplification) and circular (as in plasmid) DNA molecules are suitable for such expression as long as they contain the gene encoding the protein operably linked to an appropriate promoter. Other features of the DNA molecule that are important for optimal expression in either the bacterial or eukaryotic cells (including the ribosome binding site etc) are also included in these constructs. The proteins can again be expressed individually or in suitable size pools containing multiple library members. The main advantage offered by the in vitro systems is their speed and ability to produce soluble proteins. In addition, the protein being synthesized can be selectively labeled if needed for subsequent functional analysis.

Methods of introducing exogenous nucleic acid molecules into host cells is well-known in the art, and will vary with the host cell used. Techniques include dextran-mediated transfection, calcium phosphate precipitation, calcium chloride treatment, polybrene-mediated transfection, protoplast fusion, electroporation, viral or phage infection, encapsulation of the polynucleotide(s) in liposomes, and direct microinjection of the DNA into nuclei. In the case of mammalian cells, transfection can be either transient or stable.

A variety of recombinant expression vectors can be utilized to express the library of proteins. Examples of suitable vectors include, but are not limited to, pED (commercially available from NOVAGEN®), pBAD and pCNDA (commercially available from INVITROGEN™), pEGEX (commercially available from Amersham Biosciences), pQE (commercially available from QIAGEN®). The choice of the appropriate vector can be ascertained by one of skill in the art. Expression vectors embrace self-replicating extrachromosomal vectors or vectors which integrate into a host genome. Expression vectors used in the methods described herein typically contain a library member, control or regulatory sequences, selectable markers, and/or additional elements, such as a purification tag.

Panning and/or assays can be used to identify mutant proteins with novel functions. For example, in some embodiments, yeast two-hybrid screening methods is used to identify proteins with a desired function. Other assay methods include, but are not limited to, binding assays and activity assays. As exemplified herein, libraries are readily screened using a yeast two-hybrid system (see also Chen, et al. (2004) J. Biol. Chem. 279:33855-33864; Schwimmer, et al. (2004) Proc. Natl. Acad. Sci. USA 101:14707-14712; Doyle, et al. (2001) J. Am. Chem. Soc. 123:11367-11371). Yeast-based two-hybrid systems utilize chimeric genes and detect protein-protein interactions via the activation of reporter-gene expression. Reporter-gene expression occurs as a result of reconstitution of a functional transcription factor caused by the association of fusion proteins encoded by the chimeric genes. See also, Ausubel, et al., Current Protocols in Molecular Biology, John Wiley & Sons, pp. 13.14.1-13.14.14; Sambrook & Russell, Molecular Cloning, Cold Spring Harbor Laboratory Press, 3^{rd} edition, Chapter 18.

In addition to the yeast two-hybrid systems, other screening methods can be used to identify proteins with novel or altered functions. For example, screening methods based on cell survival, cell death, or expression of reporter genes in cells can be used. The screens can employ cells expressing individual variants or pools of variants belonging to a library.

In some embodiments, host cells other than yeast are used to identify novel proteins of interest. Suitable host cells are described herein. As exemplified herein, E. coli cells are transformed with a library representing variants of an enzyme and grown in the presence of the corresponding substrate. Only clones with a functional variant of the enzyme will survive.

In some embodiments, libraries of mutant proteins are attached to or bound to an insoluble support having isolated sample receiving areas (e.g., a microtiter plate, an array, etc.). Insoluble supports are generally made of any composition to which the assay component can be bound, are readily separated from soluble material, and are otherwise compatible with the overall method of screening. The surface of such supports can be solid or porous and of any convenient shape. Examples of suitable insoluble supports include microtiter plates, arrays, membranes and beads. These are typically made of glass, plastic (e.g., polystyrene), polysaccharides, nylon or nitrocellulose, TEFLON®, etc. Microtiter plates and arrays are especially convenient because a large number of assays can be carried out simultaneously, using small amounts of reagents and samples.

Alternatively, bead-based assays can be used, particularly when using fluorescence-activated cell sorting (FACS). The particular manner of binding the assay component is not crucial so long as it is compatible with the reagents and overall methods described herein, and maintains the activity of the composition.

The proteins of the library can be purified or isolated after expression. Library proteins can be isolated or purified in a variety of ways known to those skilled in the art depending on what other components are present in the sample. The degree of purification necessary can vary depending on the use of the library protein. In some instances no purification will be necessary. For example, in some embodiments, if library proteins are secreted, screening or selection takes place directly from the media.

Standard purification methods include electrophoretic, molecular, immunological and chromatographic techniques, including ion exchange, hydrophobic, affinity, size-exclusion chromatography, and reversed-phase HPLC chromatography, as well as precipitation, dialysis, and chromatofocusing techniques. Purification can often be facilitated by the inclusion of purification tag. The choice of the appropriate purification tag can be ascertained by one of skill in the art. For example, the library protein can be purified using glutathione resin if a GST fusion is employed, Immobilized Metal Affinity Chromatography (IMAC) if a His or other tag is employed, or immobilized anti-FLAG® antibody if a FLAG® tag is used. Ultrafiltration and diafiltration techniques, in conjunction with protein concentration, are also useful. For general guidance in suitable purification techniques, see Scopes (1994) Protein Purification: Principles and Practice, 3rd Ed., Springer-Verlag, NY.

The coevolution methods described herein are useful for generating and identifying proteins with novel or altered functions. By way of illustration, the instant method was used to generate mutants of human estrogen receptor a ligand binding domain (hERαLBD) with novel corticosterone activity. Two steroids, testosterone and progesterone were used to provide a stepwise structural bridge between 17β-estradiol (E₂) and corticosterone. Human estrogen receptor (hER) is a ligand-regulated transcription factor that mediates the actions of estrogen in different target tissues including the reproductive, pituitary, hypothalamus, bone, liver, and cardiovascular system (Katzenellenbogen, et al. (1996) Chem. Biol. 3:529-536). It is a member of the nuclear receptor superfamily that encompasses steroid receptors, non-steroid receptors, and orphan receptors (Mangelsdorf, et al. (1995) Cell 83:835-9). Like other members of the superfamily, hER has three modular structural domains, an amino-terminal ligand-independent transactivation domain, a central DNA binding domain (DBD), and a carboxy-terminal ligand binding domain (LBD). The hER LBD interacts specifically with its physiological ligand E₂ and contains a dimerization function and a ligand-independent activation function. hER has been linked with several human diseases such as breast cancer and osteoporosis, and considerable efforts have been directed at understanding the molecular basis of the estrogen receptor and ligand interactions (Katzenellenbogen, et al. (1996) supra; Mangelsdorf, et al. (1995) supra; Tenbaum & Baniahmad (1997) Int. J. Biochem. Cell Biol. 29:1325-1341; Nilsson, et al. (2001) Physiol. Rev. 81:1535-1565). Despite the low sequence homology between the LBDs of different nuclear receptors, all these proteins share a similar secondary structure of 11-12 α-helices and a small β-sheet arranged in an anti-parallel sandwich structure.

As exemplified herein, directed evolution was used to sequentially generate, hERαLBD variants that act on the two intermediates in the pathway, i.e., testosterone and progesterone. Error-prone PCR was used to introduce a low-frequency of random point mutations, approximately 1-2 amino acid substitutions per gene on average into the wild-type human ligand-binding domain (LBD) fragment encompassed by amino acids 312-595 of hERα set forth herein as SEQ ID NO:3 (Zhao, et al. (1999) In: Manual of Industrial Microbiology and Biotechnology, 2nd ed., Demain & Davies, eds. ASM Press, Washington D.C., pp. 597-604). The first and second rounds were carried out to generate first and second libraries of hERαLBD variants with increased potency to testosterone. The third and fourth rounds were used to obtain hERαLBD variants with increased potency to progesterone.

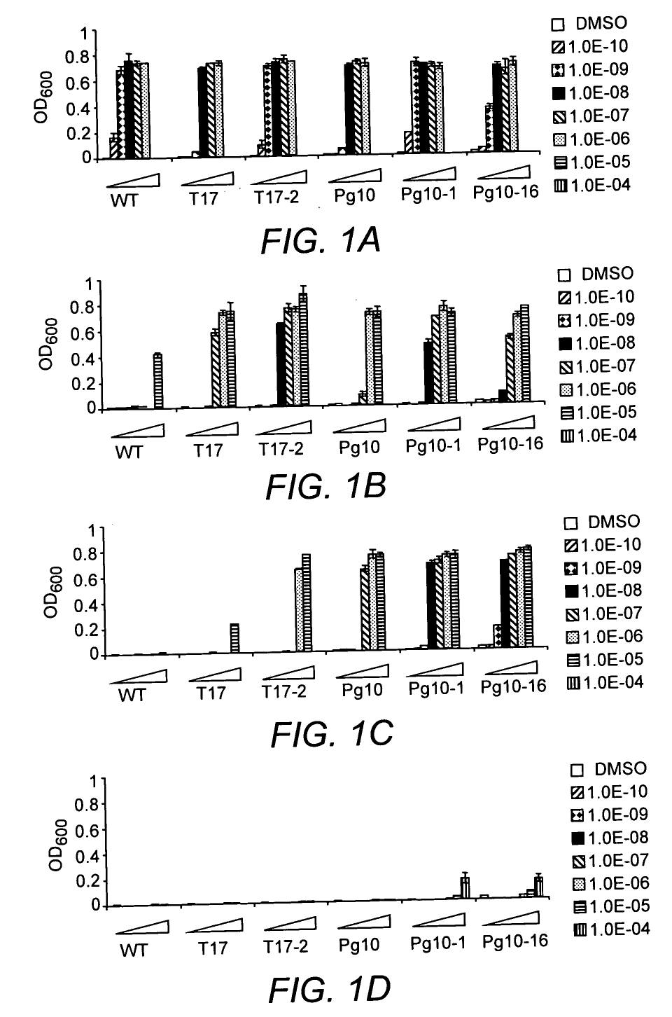

A total of approximately 10⁶ variants were screened using a yeast two-hybrid system. Screening of the first two libraries identified a hERαLBD variant, T17-2 (SEQ ID NOs:7 and 8), that showed >500-fold increased sensitivity toward testosterone in yeast compared to the wild-type hERαLBD, and also responded to progesterone at micromolar concentrations (FIG. 1). The wild-type hERαLBD had almost undetectable response to progesterone at saturating ligand concentration of 10⁻⁵ M in yeast (FIG. 1). Screening of the third and fourth libraries yielded three new variants, Pg10 (SEQ ID NOs:9 and 10), Pg10-1 (SEQ ID NOs:11 and 12) and Pg10-16 (SEQ ID NOs:13 and 14), which showed responses to progesterone at nanomolar concentrations, and responses to corticosterone (10⁻⁴ M). In comparison, the wild-type hERαLBD showed no corticosterone-dependent response in yeast.

Accordingly, the present invention also relates to mutant estrogen receptor proteins that bind testosterone and coritcosterone, as well as nucleic acid molecules, recombinant vectors, and host cells encoding and expressing the same. Suitable recombinant vectors and host cells are disclosed herein.

A nucleic acid molecule of the present invention is intended to include RNA, DNA, cDNA and the like composed of naturally occurring nucleobases, as well as analogs thereof, e.g., containing synthetic nucleobases such as 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Nucleobase polymers or oligomers can vary in size from a few nucleobases, for example, from 2 to 40 nucleobases, to several hundred nucleobases, to several thousand nucleobases, or more. Nucleobase polymer or oligomer are generally referred to herein as nucleic acid molecules.

CarA is a wild-type dioxygenase capable of deoxygenating carbazole to 2′-aminobiphenyl-2,3-diol (2′-APBD). AtdA is a multicomponent class IA dioxygenase which contains five subunits, AtdA1-A5, and is involved in the simultaneous deamination and oxygenation of aniline. AtdA can also dinitrogenate o-toluidine, which has an additional methyl side chain at the ortho position. However, AtdA cannot accept aromatic amines with ortho-position substituents larger than an ethyl group. An in vitro coevolution strategy involving a stepwise relaxation of AtdA substrate specificity is disclosed herein, wherein AtdA accepts progressively larger ortho-substituted anilines. See Scheme 4.

Mutant proteins isolated according to the methods described herein typically contain multiple mutations, that when combined in a single protein, result in a protein exhibiting novel or altered functions. For example, in some embodiments, a mutant protein containing a single mutation, or a mutant containing two or more mutations, one of which occurs at the same location, i.e., position 1, may not be capable of interacting with an analog or target molecule. In contrast, a mutant containing mutations at positions 1 and 2, is capable of interacting with an analog or target molecule. Thus, mutant proteins isolated according to the methods described herein, can contain two, three, four, five, six, seven, eight, nine, ten, or more mutations. The mutations can change the amino acid at any position within the protein. For example, one or more of the mutations can occur in the protein binding pocket, outside of the protein binding pocket, but in the protein binding domain, and/or outside of the protein binding domain. Additionally, the mutant proteins can contain additional mutations in amino acid residues that do not modify the interaction between the mutant protein and the analog or target molecule.

Thus, the coevolution methods described herein permit the generation and identification of proteins with novel functions. For example, proteins can be isolated that are capable of carrying out novel reduction reactions. As another specific example, proteins capable of carrying out novel oxidation and addition reactions can be isolated. As a further example, proteins capable of carrying out novel deamination and oxygenation reactions can be isolated.

In particular embodiments, the instant method provides for isolation of a mutant estrogen receptor alpha protein or fragment thereof, which binds two or more steroid hormones. In accord with these embodiments, the mutant protein has one more mutations in the amino acid sequence corresponding to SEQ ID NOs:12 or 14, or shares from 50% to 70% homology with another member of the estrogen receptor protein family e.g., an estrogen receptor alpha protein from Acanthopagrus schlegelii (SEQ ID NO:17), Alligator mississippiensis (SEQ ID NO:18), Astatotilapia burtoni (SEQ ID NO:19), Bos taurus (SEQ ID NO:20), Caiman crocodilus (SEQ ID NO:21), Cavia porcellus (SEQ ID NO:22), Chrysophrys major (SEQ ID NO:23), Coturnix japonica (SEQ ID NO:24), Danio rerio (SEQ ID NO:25), Equus caballus (SEQ ID NO:26), Fundulus heteroclitus (SEQ ID NO:27), Halichoeres tenuispinis (SEQ ID NO:28), Halichoeres trimaculatus (SEQ ID NO:29), Ictalurus punctatus (SEQ ID NO:30), Micropterus salmoides (SEQ ID NO:31), Mus musculus (SEQ ID NO:32), Ovis aries (SEQ ID NO:33), Oncorhynchus masou (SEQ ID NO:34), Paralichthys olivaceus (SEQ ID NO:35), Sparus aurata (SEQ ID NO:36), Taeniopygia guttata (SEQ ID NO:37), Tilapia nilotica (SEQ ID NO:38), and Xenopus laevis (SEQ ID NO:39). LBDs of these homologs are readily identified by the skilled artisan based on sequence similarities and location of the LBD in the human amino acid sequence.

In one embodiment, the mutant estrogen receptor alpha protein, or fragment thereof, binds testosterone and has mutations at residues 353 and 390 of the amino acid sequence corresponding to SEQ ID NO:2.

In another embodiment, the mutant estrogen receptor alpha protein, or fragment thereof, binds progesterone and has mutations at residues 353, 390, and 524 of the amino acid sequence corresponding to SEQ ID NO:2.

In a further embodiment, the mutant estrogen receptor alpha protein, or fragment thereof, binds corticosterone and has mutations at residues 353, 390, 524, and 536, as well as a mutation at either 528 or 585 of the amino acid sequence corresponding to SEQ ID NO:2.

The invention is described in greater detail by the following non-limiting examples.

### EXAMPLE 1

### Materials and Methods

Restriction enzymes and DNA modifying enzymes were obtained from New England BioLabs (Beverly, Mass.). Yeast strain YRG-2 (Mata ura3-52 his3-200 ade2-101 lys2-801 trp1-901 leu2-3 112 gal4-542 gal80-538 LYS2::USAGAL1-TATA GAL1-HIS3 URA3::USAGAL 4 17mers(x3)-TATACYC1-lacZ) was from STRATAGENE® (La Jolla, Calif.). Taq DNA polymerase was from PROMEGA® (Madison, Wis.). QIAPREP® spin plasmid mini-prep kit, QIAEX® II gel purification kit, and QIAQUICK® PCR purification kit were purchased from QIAGEN® (Valencia, Calif.). Various oligonucleotide primers were obtained from Integrated DNA Technologies (Coralville, Iowa). Unless otherwise specified, general chemicals were obtained from SIGMA (St. Louis, Mo.). Plasmid pBD-Gal4 hERα containing amino acids 312-595 of hERα fused to the Gal4 DNA binding domain, and plasmid pGAD424 SRC-1 containing the full length coactivator SRC-1 fused to the Gal4 activation domain were constructed as described (Chen, et al. (2004) J. Biol. Chem. 279:33855-33864).

Library construction and screening have been described (Chen, et al. (2004) supra). The third and fourth libraries of variants were screened on 5×10⁻⁸ M and 5×10⁻⁹ M progesterone, respectively.

Mutagenic PCR was performed as described (Chen, et al. (2004) supra). The average mutagenic rate was 1.7 nucleotide substitutions per gene as determined by DNA sequencing.

Single, triple and quadruple site-directed mutants were created using overlap extension PCR and yeast in vivo recombination (Chen, et al. (2004) supra). Plasmids of the different site-directed mutants were rescued from yeast cells, transferred into E. coli, and sequenced to confirm the presence of the introduced specific mutations and the absence of PCR-associated random mutations.

A yeast two-hybrid based cell growth assay was used to quantify the ligand activity of the wild-type and mutant hERαLBD in 96-well plates (Chen, et al. (2004) supra). Briefly, yeast cells harboring the plasmid containing the target hERαLBD and plasmid pGAD424 SRC-1 were grown to saturation (OD₆₀₀ 4-5) in 2-3 mL minimal medium lacking tryptophan and leucine, and then diluted to OD₆₀₀ 0.002 using minimal medium lacking tryptophan, leucine and histidine. Each well contained 200 μL diluted yeast cells and 0.2 μL of specified ligand dissolved in 100% ethanol (E₂, testosterone, and progesterone) or DMSO (corticosterone). The 96-well plates were incubated at 30° C. for 24 hours and the cell density was measured at 600 nm using a SPECTRAMAX® plate reader (Molecular Devices, Sunnyvale, Calif.).

For molecular modeling, the corticosterone ligand, generated using the Builder function of MOE (Molecular Operating Environment, Chemical Computing Group Inc., Montreal, Quebec, Canada) and energy minimized under the MMFF94s forcefield, was docked into the ligand binding pocket of human GR LBD (PDB code: 1M2Z) using the MOE Dock function. The lowest-energy docked conformation was further energy minimized. The resulting 3-dimensional structure of human GR LBD complexed with corticosterone was structurally aligned with the crystal structure of hERαLBD complexed with E₂ (PDB code: 1GWR) and imported into Visual Molecular Dynamics (VMD) (Nilsson, et al. (2001) Physiol. Rev. 81:1535-1565). Residues Glu353, Gly390, His524, and Leu536 were mutated to Gln, Asp, Asn, and His, respectively using the MOE Rotamer Explorer and the appropriate conformations were manually selected.

### EXAMPLE 2

### Corticosterone Activity of hERαLBD

The hERα has highly selective ligand specificity that enables it discriminate between different classes of steroids with closely related structures (Kuiper, et al. (1997) Endocrinology 138:863-870; Ekena, et al. (1998) J. Biol. Chem. 273:693-699). For example, although the chemical structure of testosterone (a C₁₉ steroid) and E₂ (a C₁₈ steroid) differ only slightly in the A-ring region, the activation of the hERα requires at least 10,000-fold higher concentration of testosterone relative to E₂ (Chen, et al. (2004) supra). Since corticosterone (a C₂₁ steroid) differs from E₂ in four positions in their chemical structures, it was determined whether corticosterone could bind and activate hERα.

A yeast two-hybrid-based cell growth assay was used to determine the dose-response profiles of E₂, testosterone, progesterone, and corticosterone to the wild-type hERαLBD. hERαLBD responds to sub-nanomolar concentrations of E₂, responds to testosterone only at micromolar concentrations, barely responds to progesterone at saturating ligand concentrations (˜10⁻⁵ M), and does not respond at all to corticosterone at saturating ligand concentration (˜10⁻⁴ M).

### EXAMPLE 3

### In vitro Coevolution of Novel Corticosterone Activity in hERαLBD

To create a variant of the hERαLBD that responds to corticosterone, testosterone and progesterone were used to construct an evolutionary pathway between E₂ and corticosterone (Scheme 1), and directed evolution (Dir. Evol.) was used to evolve, sequentially, hERαLBD variants that act on the two evolutionary intermediates. Steroid hormones E₂, testosterone, progesterone, and corticosterone are the physiological ligands for members of the steroid receptor family estrogen receptor (ER), androgen receptor (AR), progesterone receptor (PR), and glucocorticoid receptor (GR), respectively. In addition, these four steroid hormones are important intermediates in the biochemical pathway of cholesterol biosynthesis.

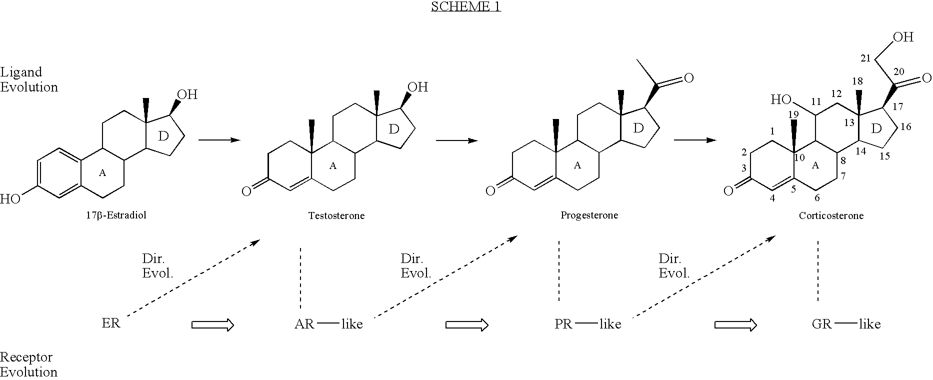

The first and second rounds of directed evolution were carried out to obtain hERαLBD variants with increased potency to testosterone, whereas the third and fourth rounds were to obtain hERαLBD variants with increased potency to progesterone. In each round, error-prone PCR was used to introduce a low-frequency of random point mutations (1-2 amino acid substitutions per gene on average (Zhao, et al. (1999) In: Manual of Industrial Microbiology and Biotechnology 2nd Edition, Demain & Davies eds., ASM Press, Washington D.C., pp. 597-604) into the ligand-binding domain (LBD) fragment composed of amino acids 312-595 of hERα (SEQ ID NO:4). A total of approximately 10⁶ variants were screened using a yeast two-hybrid system (Chen, et al. (2004) supra) (Scheme 2).

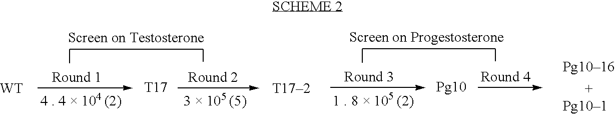

The first two rounds of directed evolution resulted in a hERαLBD variant, T17-2, that showed >500-fold increased sensitivity toward testosterone in yeast compared to the wild-type hERαLBD, and also responded to progesterone at micromolar concentrations (FIG. 1). The wild-type hERαLBD had almost undetectable response to progesterone at saturating ligand concentration of 10⁻⁵ M in yeast (FIG. 1C). The subsequent two rounds of directed evolution led to two new variants, Pg10-1 and Pg10-16 that showed responses to progesterone at nanomolar concentrations (FIG. 1C), and more importantly, showed significant responses to corticosterone (10⁻⁴ M) within 24 hours in yeast (FIG. 1D). In comparison, all the other evolved hERαLBD variants (i.e., T17, T17-2, Pg10) and the wild-type hERαLBD showed no corticosterone-dependent response in yeast, even after incubation at 30° C. for four days (FIG. 1D).

### EXAMPLE 4

### Molecular Basis Novel Corticosterone Activity

Like other members of the superfamily, hERα and hERβ have three modular structural domains, an amino-terminal ligand-independent transactivation domain, a central DNA binding domain (DBD), and a carboxy-terminal ligand binding domain (LBD)(Tables 1 and 2). The hER LBD interacts specifically with its physiological ligand E₂ and contains a dimerization function and a ligand-independent activation function.

TABLE 1

| | Position | Position Within |
|---|---|---|
| | Within HERα | HERα Coding |
| Domain | Protein^{a} | Region^{b} |
| Activation Domain 1 (AF-1) | 1-179 | 1-537 |
| DNA Binding Domain (DBD) | 180-262 | 538-786 |
| Hinge Domain | 263-301 | 787-903 |
| Ligand Binding Domain | 302-552 | 904-1656 |
| Activation Domain 2 (AF-2) | Spread out | Spread out |
| | within LBD³ | within LBD³ |
| F-Domain | 553-595 | 1657-1785 |

^{a}Position is in reference to SEQ ID NO: 2.
^{b}Position is in reference to SEQ ID NO: 1.
^{c}Nilsson et al. (2001) supra.

TABLE 2

| | Position | Position Within |
|---|---|---|
| | Within HERβ | HERβ Coding |
| Domain | Protein^{a} | Region |
| Activation Domain 1 (AF-1) | 1-143 | 1-429 |
| DNA Binding Domain (DBD) | 144-226 | 430-678 |
| Hinge Domain | 227-254 | 679-762 |
| Ligand Binding Domain | 255-504 | 763-1512 |
| Activation Domain 2 (AF-2) | Spread out | Spread out |
| | within LBD^{b} | within LBD^{b} |
| F-Domain | 505-530 | 1513-1590 |

^{a}Position is in reference to SEQ ID NO: 6.
^{b}Position is in reference to SEQ ID NO: 5.
^{c}Nilsson et al. (2001) supra.

To identify the molecular basis for the creation of this novel ligand activity, five evolved variants were sequenced. Seven non-synonymous mutations were identified (Table 3).

TABLE 3

| hERα | |
|---|---|
| Variant | Amino Acid Substitutions |
| T17 | Glu353Gln |
| T17-2 | Glu353Gln, Gly390Asp |
| Pg10 | Glu353Gln, Gly390Asp, His524Asn |
| Pg10-1 | Glu353Gln, Gly390Asp, His524Asn, |
| | Leu536His, Thr585Ser |
| Pg10-16 | Glu353Gln, Gly390Asp, His524Asn, |
| | Met528Leu, Leu536Pro |

Mutation Glu353Gln was located in the ligand binding pocket and altered the hydrogen-bonding pattern near the A-ring of the ligand between the receptor and the ligand. Glu353 (a hydrogen bond acceptor) pairs well with the 3-phenolic group of E₂ (a hydrogen bond donor), whereas Gln353 (a hydrogen bond donor) pairs well with the 3-keto group of testosterone, progesterone, or corticosterone (a hydrogen bond acceptor). Mutation Glu353Gln accounts for the emergence of ligand activity of the evolved hERαLBD variants toward 3-ketosteroids as residue Gln353 is conserved in androgen receptors, progesterone receptors, glucocorticoid receptors, and mineralocorticoid receptors.

Mutation Gly390Asp was not within the ligand binding pocket. Molecular modeling indicated that this mutation formed a new electrostatic interaction with Arg394 to compensate for the loss of the electrostatic interaction formed between Glu353 and Arg394 in the wild-type hERαLBD, thus stabilizing the overall interactions between the receptor and the ligand.

Mutation His524Asn appeared to abolish the hydrogen bond formed between the δ-nitrogen of histidine and the 17β-hydroxyl group of E₂, while establishing a new hydrogen bond between the 20-keto group of progesterone or corticosterone and the γ-amino group of asparagine. In this regard, Pg10 (Glu353Gln, Gly390Asp, His524Asn) showed ˜10-fold higher sensitivity to progesterone (FIG. 1C), and ˜10-fold and ˜50-fold lower sensitivity to E₂ (FIG. 1A) and testosterone (FIG. 1B), respectively, than T17-2 (Glu353Gln, Gly390Asp). Similarly, the single mutant His524Asn showed a slightly increased sensitivity to progesterone (FIG. 1C), and ˜5-fold decreased sensitivity to E₂ (FIG. 1A) compared to the wild-type hERαLBD.

None of the three selected variants from the first three rounds of directed evolution (i.e., T17, T17-2, and Pg10) showed any response to corticosterone (FIG. 1D); only the two fourth-round variants, Pg10-1 and Pg10-16, responded to corticosterone at submillimolar concentrations in yeast cells. In comparison with Pg10, both Pg10-1 and Pg10-16 contained two additional mutations, with one occurring at the same position (Leu536). Four quadruple mutants, Pg10+Leu536His, Pg10+Leu536Pro, Pg10+Thr585Ser, and Pg10+Met528Leu were created by site-directed mutagenesis and assayed for their transactivation activity in yeast cells. All of these quadruple mutants showed increased sensitivity to progesterone, but only the first three (i.e., Pg10+Leu536His, Pg10+Leu536Pro, Pg10+Thr585Ser) showed response to corticosterone. The ligand binding affinities of the wild-type and mutant hERαLBD proteins are shown in Table 4.

TABLE 4

| Estrogen | K_{d}^{E2} | RBA^{b} | | | K_{d} (nM)^{c} | | |
|---|---|---|---|---|---|---|---|
| Receptor | (nm)^{a} | T^{d} | Pg | Cs | T | Pg | Cs |
| Wild-type | 0.21 ± 0.12 (3) | <10⁻⁴ (2) | <10⁻⁴ (2) | <10⁻⁴ (2) | | | |
| T17 | 1.01 ± 0.44 (2) | 0.52 ± 0.18 (2) | 0.016 ± 0.014 (2) | <10⁻⁴ (2) | 193 | 6334 | |
| T17-2 | 0.31 ± 0.13 (2) | 0.97 ± 0.20 (3) | 0.017 ± 0.005 (3) | <10⁻³ (2) | 32 | 1801 | |
| Pg10 | 1.28 ± 0.09 (2) | 0.15 ± 0.08 (2) | 0.674 ± 0.063 (2) | 0.017 ± 0.002 (2) | 832 | 191 | 7612 |
| Pg10-1 | 0.81 ± 0.23 (3) | 0.21 ± 0.06 (3) | 0.679 ± 0.021 (3) | 0.008 ± 0.001 (3) | 388 | 119 | 10288 |
| Pg10-16 | 2.95 ± 0.27 (2) | 0.10 ± 0.03 (3) | 2.184 ± 1.3 (3) | 0.015 ± 0.009 (3) | 2872 | 135 | 20227 |

^{a}K_{d}^{E2} values were determined by Scatchard analysis from multiple independent experiments (n = 2-3), and the error bounds represent the range (n = 2) or S.E. (n > 2).
^{b}RBA values were determined with 2 nM [³H]-E₂ for wild-type and all mutants. RBA = EC₅₀^{E2}/EC₅₀^{ligand} × 100. Values represent the average of multiple independent determinations (n = 2-3).
^{c}The binding affinity of testosterone, progesterone or corticosterone was calculated with K_{d}^{ligand} = (K_{d}^{E2}/RBA) × 100.
^{d}T = testosterone, Pg = progesterone, and Cs = corticosterone.

None of these four mutations were within the ligand binding pocket. Residue Leu536 is located in the loop connecting helix 11 and helix 12, and is thought to be critical in coupling the binding of ligand to the modulation of the conformation and activity of the hERα (Zhao, et al. (2003) J. Biol. Chem. 278:27278-27286). The functions of both Leu536His and Leu536Pro were context-dependent, as quadruple mutants Pg10+Leu536His and Pg10+Leu536Pro showed no or negligible ligand-independent response, whereas single mutants Leu536His and Leu536Pro showed significantly elevated ligand-independent response in yeast cells.

Molecular modeling indicated that Leu525 (located on helix 11) formed a van der Waals interaction with Leu536, and unlike its corresponding residue in human glucocorticoid receptor (Cys736), Leu525 sterically clashed with the larger substituent at the C17α position of corticosterone compared with the corresponding substituent in E₂, testosterone or progesterone. Thus, the substitution of Leu536 by a residue with a smaller side chain (Leu536His or Leu536Pro) likely shifts the side chain position of Leu525, resulting in a larger side pocket of hERα near the C17 atom of E₂ to accommodate the large substituent at the C17α position of corticosterone. Mutation Thr585Ser was not located in the ligand binding domain, and its effect on ligand binding was unclear.

None of the single mutants containing each of the seven mutations showed any response to corticosterone in yeast cells. In addition, using corticosterone as a selection ligand, two hERαLBD libraries (3.7×10⁶ variants per library) created by error-prone PCR with low and high mutagenesis rates (1.7 and 11 nucleotide substitutions per gene, respectively) were screened and failed to identify any mutants responding to corticosterone. Furthermore, triple mutants (Glu353Gln+Gly390Asp+Leu536His, Glu353Gln+Gly390Asp+Leu536Pro, Glu353Gln+Gly390Asp+Thr585Ser, and Glu353Gln+Gly390Asp+Met528Leu) did not show any corticosterone-dependent response. Thus, the creation of corticosterone activity in the wild-type hERαLBD using the yeast two-hybrid system-based screening method required at least four simultaneous mutations. Although these changes could not be obtained directly by a one-step directed evolution approach, the desired activity was efficiently achieved using the progressive ligand-receptor coevolution strategy disclosed herein.

These results provide insight into the molecular evolution of nuclear steroid receptors. There are six evolutionarily-related steroid receptors that have been discovered, including estrogen receptors α and β (ERα and ERβ), progesterone receptor, androgen receptor, glucocorticoid receptor, and mineralocorticoid receptor. Molecular phylogenetic analysis suggests that all steroid receptors have evolved from an ancestral estrogen receptor through a series of gene duplication and divergent evolution (Laudet (1997) J. Mol. Endocrinol. 19:207-226). A ligand exploitation model was proposed as an evolutionary mechanism for the creation of a novel ligand-receptor pair. New hormones emerged when duplicated receptors evolved increased affinity for biochemical intermediates in a biosynthetic pathway (Thornton (2001) Proc. Natl. Acad. Sci. USA 98:5671-5676; Thornton, et al. (2003) Science 301:1714-1717).

Consistent with such a model, the instant results indicate that a novel corticosterone activity can be readily created in the laboratory by coevolving the estrogen receptor and biochemical intermediates including testesterone and progesterone from the cholesterol biosynthetic pathway. However, unlike the naturally occurring steroid receptors, the laboratory-evolved hERα variants are promiscuous receptors, suggesting both positive and negative selection forces may operate simultaneously in nature.

The described in vitro coevolution approach has advantages over rational design and directed evolution approaches. Although structure-based computational design allows a vast number of protein variants to be screened in silico (>10¹⁴), the search for mutations is limited to a particular region, i.e., residues forming direct contacts with the substrate or the ligand (Hayes, et al. (2002) Proc. Natl. Acad. Sci. USA 99:15926-15931; Looger, et al. (2003) Nature 423:185-190). As shown herein and by others (Yano, et al. (1998) Proc. Natl. Acad. Sci. USA 95:5511-5515; Chen, et al. (2004) supra; Nettles, et al. (2004) Mol. Cell 13:317-3278-10), residues far away from the enzyme active site or ligand-binding pocket can exert their effects on protein functions through long-range interactions whose analysis is still beyond the capability of existing computational design approaches.

Directed evolution generally requires a screening or selection method to detect the target function in the wild-type protein. The power of directed evolution is limited by the number of sequences (library size) that can be screened experimentally (about 10¹⁴ for library panning and 10⁷ for high throughput screening) (Hayes, et al. (2002) supra). Thus, directed evolution is useful for fine-tuning the protein function, but is not especially well-suited for creating novel functions that may require multiple simultaneous mutations. In contrast, in vitro coevolution allows the target novel function to be divided into a few intermediate functions that are amenable to classical directed evolution. Since single or double mutations can show beneficial effects in these intermediate functions, only a small library of protein variants (less than 10⁴⁻⁵) need to be screened in each round of directed evolution. The accumulation of these beneficial mutations eventually leads to the creation of the target novel functions.

### EXAMPLE 5

### Novel Carbazole Denitrogenation Pathway by In Vitro Coevolution Engineering

Aromatic-nitrogen compounds are currently removed from petroleum using high pressure or high temperature hydrotreating processes. However, these processes are hazardous, expensive, and can modify other constituents of petroleum. The use of microorganisms to degrade carbazole offers a more environmentally friendly and cost-effective alternative to current industrial denitrogenation methods used. Current carbazole-degrading pathways, such as the Car operon of Pseudomonas strain CA103, incorporate the degradation products into the biomass of the microorganism. This results in the loss of most of the fuel value of carbazole.

Generation of a novel carbazole denitrogenation pathway by combining two enzymes, carbazole-1,9a-dioxygenase (CarA) and an aniline dioxygenase (AtdA) mutant in E. coli (Sato, et al. (1997) J. Bacteriol. 179:4850-4858) provides an alternative to bacterial degradation. In this pathway, carbazole is first dioxygenated into 2′-aminobiphenyl-2,3-diol (2′-ABPD) by CarA. Subsequently, the amine group from 2′-ABPD is removed by the AtdA enzyme via a dioxygenation reaction. This pathway is shown in Scheme 3.

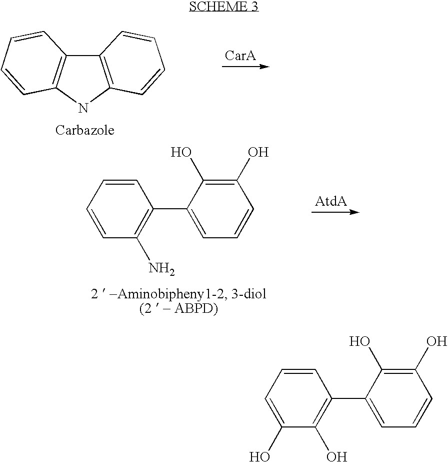

AtdA is a multicomponent class IA dioxygenase isolated from Acinetobacter sp. strain YAA5. It contains five subunits, AtdA1-A5 and is involved in the simultaneous deamination and oxygenation of aniline. AtdA can also dinitrogenate o-toluidine, which has an additional methyl side chain at the ortho position (Takeo, et al. (1998) J. Ferment. Bioengin. 85:514-517). 2′-ABPD can be viewed as an aniline molecule with a bihydroxylated phenyl attached at the ortho position. However, 2′-ABPD is not a substrate for AtdA because AtdA does not accept aromatic amines with ortho-position substituents larger than an ethyl group.

Scheme 4 illustrates an in vitro coevolution strategy involving a stepwise relaxation of the AtdA3 enzyme's substrate specificity to accept progressively larger ortho-substituted aniline. AtdA3 is believed to be the subunit of a terminal dioxygenase (Takeo, et al. (1998) supra), which determines the substrate specificity of the enzyme (Butler & Mason (1997) Structure-function analysis of the bacterial aromatic ring-hydroxylating dioxygenases, Advances in Microbial Physiology, Vol. 38, Elsevier Science & Technology Books).

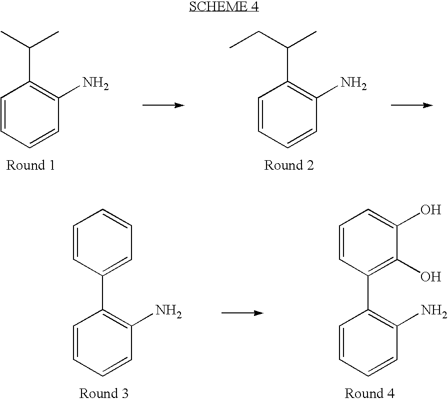

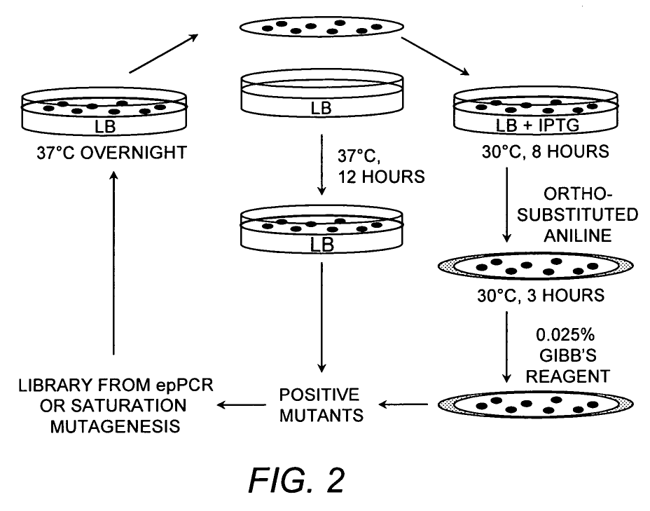

A library of AtdA3 mutants is created by error prone PCR or saturation mutagenesis of the binding pocket residues identified from homology modeling. These mutants are screened for the ability to denitrogenate a specific ortho-substituted aniline, i.e., Round 1 in Scheme 4, using the Gibb's reagent solid phase screen (Joern, et al. (2001) J. Biomol. Screen 6:219-223) (FIG. 2). Positive clones are selected and put through another round of directed evolution using an ortho-substituted aniline with a sterically larger ortho-substituent, i.e., Round 2, in Scheme 4. This process is repeated several more times using progressively larger ortho-substituted anilines (see, e.g., Scheme 4), until a variant of AtdA3 is isolated that uses 2′-ABPD as a substrate.

### EXAMPLE 6

### Novel Homing Endonuclease by In Vitro Coevolution Engineering

Homing endonuclease genes are mobile DNA elements that are encoded by introns and inteins. They reside within the host genomes of all three biological kingdoms and can promote a site-specific double-strand break in intron-less or intein-less alleles to facilitate the homing of their respective genetic elements (Belfort & Perlman (1995) J. Biol. Chem. 270(51):30237-40; Curcio & Belfort (1996) Cell 84(1):9-12; Cooper & Stevens (1995) Trends Biochem. Sci. 20(9):351-6). Such site-specificity arises from the ability of the endonucleases to recognize and cleave long DNA sequences (14-40 bp). Based on their structural and functional similarities, homing endonucleases that initiate the mobility process can be grouped into four families, LAGLIDADG, GIY-YIG, H-N-H and His-Cys box (Belfort & Perlman (1995) J. Biol. Chem. 270(51):30237-40). LAGLIDADG makes up the largest family and contains several hundred identified members, many of which are functional endonucleases (Dalgaard, et al. (1997) Nucleic Acids Res. 25(22):4626-38). LAGLIDADG enzymes contain one or two Leu-Ala-Gly-Leu-Ile-Asp-Ala-Asp-Gly (SEQ ID NO:40) motifs, which form the dimer interface between endonuclease domains or subunits and contribute conserved acidic residues to the enzyme active sites (Duan, et al. (1997) Cell 89(4):555-64; Heath, et al. (1997) Nat. Struct. Biol. 4(6):468-76). Endonucleases with a single Leu-Ala-Gly-Leu-Ile-Asp-Ala-Asp-Gly (SEQ ID NO:40) motif form homodimers that recognize palindromic or pseudo-palindromic DNA target sites while members containing two motifs per polypeptide chain fold to form pseudo-symmetric monomers capable of recognizing DNA target sites with significant asymmetry (Cohen-Tannoudji, et al. (1998) Mol. Cell Biol. 18(3):1444-8).

Variants of homing endonucleases exhibiting sequence specificity for DNA sequences are of interest in gene therapy. In vitro coevolution is used to obtain variants of existing homing endonucleases with altered or novel DNA sequence specificity. For example, during each round of directed evolution, 1-2 bases of the original DNA target sequence is mutated and homing endonuclease variants with increased catalytic efficiency toward the new target sequence are selected. This process is repeated until the original DNA target sequence is completely converted into the new DNA target sequence and a homing endonuclease variant with the desired sequence specificity is obtained.

For example, homing endonuclease variants useful for treating glaucoma (GLC1A) are generated using the methods described herein. Specifically, a novel homing endonuclease is generated that can make a specific double-strand break within the GLC1A gene thereby facilitating its replacement by a healthy gene. A target sequence for engineering a novel homing endonuclease is identified by aligning the sequence of GLC1A with the wild-type target sequence of homing endonuclease I-SceI. The target sequence of wild type I-SceI is 5′-TAG GGA TAA CAG GGT AAT-3′ (SEQ ID NO:41) (Table 5). An exemplary new target sequence located in the GLC1A gene is 5′-CAG GGG GAG CTG GGC ACC-3′ (SEQ ID NO:42). The new target sequence contains 9 base-pairs that are different from the wild-type target sequence of homing endonuclease I-SceI. The target sequence is mutated 1-2 bases in several rounds of directed evolution to obtain a variant I-SceI with the desired target sequence specificity (Table 5).

TABLE 5

| | |
|---|---|
| Round of | Changes to Wild-Type I-SceI |

| | | |
|---|---|---|
| Coevolution | Target Sequence | SEQ ID NO: |

| | | |
|---|---|---|
| 1 | TAGGGATAACAGGGCAAT | 43 |
| 2 | TAGGGGGAACAGGGCAAT | 44 |
| 3 | TAGGGGGAGCTGGGCAAT | 45 |
| 4 | TAGGGGGAGCTGGGCACC | 46 |
| 5 | CAGGGGGAGCTGGGCACC | 42 |

To identify variant I-SceI with the desired target sequence specificity, a screening system for the selection of desired homing endonuclease variants is employed. While several approaches have been reported to assay DNA cleavage event in vitro (Li, et al. (2000) Nucleic Acids Res. 28(11):E52; Lee & Han (1997) Methods Enzymol. 278:343-63; Ason & Reznikoff (2004) Nucleic Acids Res. 32(10):E83), few provide an efficient assay system in vivo (Seligman, et al. (1997) Genetics 147(4):1653-64), and even less can be used in a directed evolution experiment (Gruen, et al. (2002) Nucleic Acids Res. 30(7):E29).

Accordingly, a screening strategy is developed that links DNA cleavage by the homing endonuclease variants to the survival of E. coli transformed with genes encoding the homing endonuclease variants. The screening system takes advantage of the ability of homing endonucleases to transform circular DNA into linear product. Since linear DNA is rapidly degraded in E. coli by the endogenous RecBCD nuclease (Kuzminov & Stahl (1997) J. Bacteriol. 179(3):880-8), the endonuclease-catalyzed DNA cleavage of a plasmid containing a toxin gene results in cell survival. The system requires two plasmids, a reporter plasmid encoding a toxin gene and a homing endonuclease plasmid. A toxic gene such as ccdB (Bahassi, et al. (1995) Mol. Microbiol. 15 (6):1031-7; Loris, et al. (1999) J. Mol. Biol. 285(4):1667-77) is placed under the control of the pBAD promoter. The desired homing endonuclease target site is cloned in front of ccdB to ensure high sensitivity (Kuzminov & Stahl (1997) supra). The reporter plasmid also contains an arabinose transporter gene LacY under control of the catabolite-insensitive lacUV5 promoter. Homing endonuclease I-SceI is cloned under the control of a lacUV5 promoter on an homing endonuclease plasmid.

To cage the toxicity of the toxin, the ccdB gene is placed under the control of the pBAD promoter and transformed into a ΔcyaA E. coli strain. The pBAD promoter is known for its tight regulation by arabinose and cAMP (William (1999) Concepts of Genetics, 6th ed., Prentice Hall. 900) and having a high induction ratio among all known inducible promoters (Guzman, et al. (1995) J. Bacteriol. 177(14):4121-30). Transformation of the reporter plasmid into a wild-type E. coli strain results in low cell survival. Cell growth defects are not observed in the ΔcyaA strain transformed with the same plasmid.

Toxin gene expression is induced by the addition of cAMP (1 mg/mL) and L-arabinose (10 mM) into the liquid culture, with 99.95% of the ΔcyaA population being eliminated within 30 minutes. The 0.05% of cell survival is believed to be due to the inaccessibility of L-arabinose to the cytoplasm. The induction of pBAD promoter requires that the inducer arabinose to be transported into the cell by transporter proteins, which are also under the control of pBAD promoter. This autocatalytic behavior of pBAD promoter results in “all-or-none” gene expression (Smolke, et al. (2001) Appl. Microbiol. Biotechnol. 57(5-6):689-96). An additional arabinose transporter, LacY (Morgan-Kiss, et al. (2002) Proc. Natl. Acad. Sci. USA 99(11):7373-7) under a different promoter, lacUV5, is introduced to ensure the transport of arabinose into the cell.

To identify variants, the ΔcyaA E. coli strain is transformed with the reporter plasmid and the homing endonuclease plasmid. The homing endonuclease plasmid contains the homing endonuclease I-SceI library created by error-prone PCR and the reporter plasmid encodes the desired new target sequence. The expression of 1-SceI is induced first with IPTG and I-SceI variants catalyze the DNA cleavage at the desired target site and linearize the reporter plasmid. IPTG also induces the expression of arabinose transporter gene LacY. The linearized plasmid is then quickly eliminated from the cell and prevents the expression of toxin ccdB upon secondary induction by arabinose and cAMP, while unlinearized reporter results in the toxin expression and cell death. In this regard, the cell survival event is linked to the DNA cleavage event and homing endonuclease variants with desired DNA specificity are selected.

What is claimed is:

1. A method for identifying a mutant protein which interacts with a target molecule comprising
a) designing from a base molecule, which interacts with a known protein, a target molecule and at least one analog molecule, wherein the analog molecule represents a structural intermediate between the base molecule and the target molecule;
b) generating a first library of mutant proteins;
c) identifying from the first library of mutant proteins at least one mutant protein that interacts with the analog molecule;
d) generating from the first mutant protein a second library of mutant proteins; and
e) identifying from the second library of mutant proteins at least one mutant protein that interacts with the target molecule so that a mutant protein that interacts with the target molecule is identified.

2. An isolated mutant protein identified by the method of claim 1.

3. The isolated mutant protein of claim 2, wherein said protein is a mutant estrogen receptor alpha which binds two or more steroid hormones.

4. An isolated polynucleotide or fragment thereof, encoding the mutant protein of claim 2.

5. A recombinant vector comprising an isolated polynucleotide or fragment thereof, encoding the mutant protein of claim 2.

6. A host cell comprising an isolated polynucleotide or fragment thereof, encoding the mutant protein of claim 2.

7. A method for generating a mutant protein which interacts with a target molecule comprising
designing from a base molecule, which interacts with a known protein, a target molecule and at least one analog molecule, wherein the analog molecule represents a structural intermediate between the base molecule and the target molecule; and
sequentially performing directed coevolution on the known protein so that at least one mutant protein is generated that binds to the analog molecule and target molecule.
